SEQUENCE LISTING (41 sequences)

SEQ ID NO: 1 - protein, 497 aa, synthetic construct
MAVVYVISKSGKPLMPTTRCGHVRILLKEGKARVVERKPFTIQLTYESAEETQPLVLGIDPGRTNIGMSVVTESGESVFNAQIETRNKDVPKLMKDRKQYRMAHRRLKRRCKRRRRAKAAGTAFEEGEKQRLLPGCFKPITCKSIRNKEARFNNRKRPVGWLTPTANHLLVTHLNVVKKVQKILPVAKVVLELNRFSFMAMNNPKVQRWQYQRGPLYGKGSVEEAVSMQQDGHCLFCKHGIDHYHHVVPRRKNGSETLENRVGLCEEHHRLVHTDKEWEANLASKKSGMNKKYHALSVLNQIIPYLADQLADMFPGNFCVTSGQDTYLFREEHGIPKDHYLDAYCIACSALTDAKKVSSPKGRPYMVHQFRRHDRQACHKANLNRSYYMGGKLVATNRHKAMDQKTDSLEEYRAAHSAADVSKLTVKHPSAQYKDMSRIMPGSILVSGEGKLFTLSRSEGRNKGQVNYFVSTEGIKYWARKCQYLRNNGGLQIYVGS

SEQ ID NO: 2 - protein, 443 aa, synthetic construct
LVLGIDPGRTNIGMSVVTESGESVFNAQIETRNKDVPKLMKDRKQYRMAHRRLKRRCKRRRRAKAAGTAFEEGEKQRLLPGCFKPITCKSIRNKEARFNNRKRPVGWLTPTANHLLVTHLNVVKKVQKILPVAKVVLELNRFSFMAMNNPKVQRWQYQRGPLYGKGSVEEAVSMQQDGHCLFCKHGIDHYHHVVPRRKNGSETLENRVGLCEEHHRLVHTDKEWEANLASKKSGMNKKYHALSVLNQIIPYLADQLADMFPGNFCVTSGQDTYLFREEHGIPKDHYLDAYCIACSALTDAKKVSSPKGRPYMVHQFRRHDRQACHKANLNRSYYMGGKLVATNRHKAMDQKTDSLEEYRAAHSAADVSKLTVKHPSAQYKDMSRIMPGSILVSGEGKLFTLSRSEGRNKGQVNYFVSTEGIKYWARKCQYLRNNGGLQIYVGS

SEQ ID NO: 3 - protein, 16 aa, synthetic construct
AAAAGAGUGAACGAGA

SEQ ID NO: 4 - DNA, 22 bp, synthetic construct
  source 1..22: mol_type = other DNA
aagatcattttttttgagaaaa

SEQ ID NO: 5 - protein, 54 aa, synthetic construct
MAVVYVISKSGKPLMPTTRCGHVRILLKEGKARVVERKPFTIQLTYESAEETQP

SEQ ID NO: 6 - protein, 495 aa, synthetic construct
PLVLGIDPGRTNIGMSVVTESGESVFNAQIETRNKDVPKLMKDRKQYRMAHRRLKRRCKRRRRAKAAGTAFEEGEKQRLLPGCFKPITCKSIRNKEARFNNRKRPVGWLTPTANHLLVTHLNVVKKVQKILPVAKVVLELNRFSFMAMNNPKVQRWQYQRGPLYGKGSVEEAVSMQQDGHCLFCKHGIDHYHHVVPRRKNGSETLENRVGLCEEHHRLVHTDKEWEANLASKKSGMNKKYHALSVLNQIIPYLADQLADMFPGNFCVTSGQDTYLFREEHGIPKDHYLDAYCIACSALTDAKKVSSPKGRPYMVHQFRRHDRQACHKANLNRSYYMGGKLVATNRHKAMDQKTDSLEEYRAAHSAADVSKLTVKHPSAQYKDMSRIMPGSILVSGEGKLFTLSRSEGRNKGQVNYFVSTEGIKYWARKCQYLRNNGGLQIYVMAVVYVISKSGKPLMPTTRCGHVRILLKEGKARVVERKPFTIQLTYESAEETQ

SEQ ID NO: 7 - protein, 15 aa, synthetic construct
GGGGSGGGGSGGGGS

SEQ ID NO: 8 - protein, 596 aa, synthetic construct
MGWSHPQFEKGGGSGGGSGGSAWSHPQFEKSDLEVLFQGPLGSPKKKRKVGSKRPAATKKAGQAKKKKGSPKKKRKVGGGGSPLVLGIDPGRTNIGMSVVTESGESVFNAQIETRNKDVPKLMKDRKQYRMAHRRLKRRCKRRRRAKAAGTAFEEGEKQRLLPGCFKPITCKSIRNKEARFNNRKRPVGWLTPTANHLLVTHLNVVKKVQKILPVAKVVLELNRFSFMAMNNPKVQRWQYQRGPLYGKGSVEEAVSMQQDGHCLFCKHGIDHYHHVVPRRKNGSETLENRVGLCEEHHRLVHTDKEWEANLASKKSGMNKKYHALSVLNQIIPYLADQLADMFPGNFCVTSGQDTYLFREEHGIPKDHYLDAYCIACSALTDAKKVSSPKGRPYMVHQFRRHDRQACHKANLNRSYYMGGKLVATNRHKAMDQKTDSLEEYRAAHSAADVSKLTVKHPSAQYKDMSRIMPGSILVSGEGKLFTLSRSEGRNKGQVNYFVSTEGIKYWARKCQYLRNNGGLQIYVGGGGSGGGGSGGGGSGGGGMAVVYVISKSGKPLMPTTRCGHVRILLKEGKARVVERKPFTIQLTYESAEETQ

SEQ ID NO: 9 - RNA, 222 nt, synthetic construct
  source 1..222: mol_type = other RNA
nnnnnnnnnnnnnnnnggctcttccaactttatggttgcgaccgtaggttgaaagagcacaggctgagacattcgtaaggccgaaagaccggacgcaccctgggatttccccagtccccggaactgcatagcggatgccagttgatggagcaatctatcagataagccagggggaacaatcacctctctgtatcagagagagttttacaaaaggaggaacgg

SEQ ID NO: 10 - RNA, 36 nt, synthetic construct
  source 1..36: mol_type = other RNA
nnnnnnnnnnnnnnnnggctcttccaactttatggt

SEQ ID NO: 11 - RNA, 186 nt, synthetic construct
  source 1..186: mol_type = other RNA
tgcgaccgtaggttgaaagagcacaggctgagacattcgtaaggccgaaagaccggacgcaccctgggatttccccagtccccggaactgcatagcggatgccagttgatggagcaatctatcagataagccagggggaacaatcacctctctgtatcagagagagttttacaaaaggaggaacgg

SEQ ID NO: 12 - RNA, 222 nt, synthetic construct
  source 1..222: mol_type = other RNA
aaaagagtgaacgagaggctcttccaactttatggttgcgaccgtaggttgaaagagcacaggctgagacattcgtaaggccgaaagaccggacgcaccctgggatttccccagtccccggaactgcatagcggatgccagttgatggagcaatctatcagataagccagggggaacaatcacctctctgtatcagagagagttttacaaaaggaggaacgg

SEQ ID NO: 13 - DNA, 412 bp, synthetic construct
  source 1..412: mol_type = other DNA
  misc_feature 1..60: note = This segment is DNA
  misc_feature 61..131: note = This segment is RNA
  misc_feature 132..135: note = This segment is DNA
  misc_feature 136..155: note = This segment is RNA
  misc_feature 156..412: note = This segment is DNA
cgccccatcaaaaaaatattgacaacataaaaaactttgtgtaatacttgtaacgctgggtgtcaggcctgctagtcagccacagcttggggaaagctgtgcagcctgtgacccccccaggagaagctgggaattatcaaaaagagtgaacgagaggctctttcaacttgaaaaggttgaaagagcacaggctgagacattcgtaaggccgaaaggccggacgcaccctgggatttccccagtccccggaactgcatagcggatgtcagttgatcggccgagtaatttacgtcgacgttgacgtcgatggttgcggccgatcagataagccagggggaacaatctttcagacttggatccctctttttgatgatgctttgccgatccttagcgaaagctaaggatttttttt

SEQ ID NO: 14 - DNA, 322 bp, synthetic construct
  source 1..322: mol_type = other DNA
cgccccatcaaaaaaatattgacaacataaaaaactttgtgtaatacttgtaacgctgaaaagagtgaacgagaggctctttcaacttgaaaaggttgaaagagcacaggctgagacattcgtaaggccgaaaggccggacgcaccctgggatttccccagtccccggaactgcatagcggatgtcagttgatcggccgagtaatttacgtcgacgttgacgtcgatggttgcggccgatcagataagccagggggaacaatcacctctctggaaacagagagagttttttttatccttagcgaaagctaaggatttttttt

SEQ ID NO: 15 - protein, 23 aa, synthetic construct
GNAKTRRHERRRKLAIERDTIGY

SEQ ID NO: 16 - RNA, 235 nt, synthetic construct
  source 1..235: mol_type = other RNA
aaaagagtgaacgagaggctctttcaacttgaaaaggttgaaagagcacaggctgagacattcgtaaggccgaaaggccggacgcaccctgggatttccccagtccccggaactgcatagcggatgtcagttgatcggccgagtaatttacgtcgacgttgacgtcgatggttgcggccgatcagataagccagggggaacaatcacctctctggaaacagagagagtttttttt

SEQ ID NO: 17 - protein, 16 aa, synthetic construct
KRPAATKKAGQAKKKK

SEQ ID NO: 18 - protein, 7 aa, synthetic construct
PKKKRKV

SEQ ID NO: 19 - DNA, 21 bp, synthetic construct
  source 1..21: mol_type = other DNA
ctggttctgggtattgatccg

SEQ ID NO: 20 - DNA, 19 bp, synthetic construct
  source 1..19: mol_type = other DNA
agatcccaccttccgtttc

SEQ ID NO: 21 - DNA, 60 bp, synthetic construct
  source 1..60: mol_type = other DNA
  modified_base 25..32: mod_base = OTHER; note = Nucleotides are attached to adjacent nucleotides via a phosphorothioate bond
cgccccacgagggtacggcaaaagagttttttttactagaagtcgaggtcagcccgtggc

SEQ ID NO: 22 - DNA, 60 bp, synthetic construct
  source 1..60: mol_type = other DNA
  modified_base 26..32: mod_base = OTHER; note = Nucleotides are attached to adjacent nucleotides via a phosphorothioate bond
gccacgggctgacctcgacttctagtctcgttcactcttttgccgtaccctcgtggggcg

SEQ ID NO: 23 - DNA, 78 bp, synthetic construct
  source 1..78: mol_type = other DNA
cccacgaagggttacggcaaagcatcatcaaaaagagtgaacgagactagaagtctgaaaaggtcattttttaaagcc

SEQ ID NO: 24 - DNA, 59 bp, synthetic construct
  source 1..59: mol_type = other DNA
ccgcaagaggatgattcgggtgcggcaacggaaggggagggccccacgaagggttacgg

SEQ ID NO: 25 - DNA, 55 bp, synthetic construct
  source 1..55: mol_type = other DNA
gctgatctgatgcagttaagtgcctgctgggctttaaaaaatgaccttttcagac

SEQ ID NO: 26 - DNA, 15 bp, synthetic construct
  source 1..15: mol_type = other DNA
gactagaagtcgagg

SEQ ID NO: 27 - DNA, 29 bp, synthetic construct
  source 1..29: mol_type = other DNA
cctcgacttctagtctcgttcactctttt

SEQ ID NO: 28 - RNA, 208 nt, synthetic construct
  source 1..208: mol_type = other RNA
aaaagagtgaacgagaggctcttccaacttnnnnnnnnnnnnnnnaggttgaaagagcacaggctgagacattcgtaaggccgaaggaccggacgcaccctgggatttccccagtccccggaactgcatagcggatgccagttgatnnnnnnnnnnatcagataagccagggggaacaatcacctctctgtatcagagagagttttac

SEQ ID NO: 29 - protein, 34 aa, synthetic construct
HYHHVVPRRKNGSETLENRVGLCEEHHRLVHTDK

SEQ ID NO: 30 - protein, 34 aa, synthetic construct
HYHHVNPRHRNGSETLENRAGLCKEHHFLVHTEE

SEQ ID NO: 31 - protein, 34 aa, synthetic construct
QIEHIRPKSAGGSNRLSNLTLACAPCNHKKGAQS

SEQ ID NO: 32 - protein, 34 aa, synthetic construct
EVHHIIFRSRNGSDEEANLLTLCKTCHDGLHAGT

SEQ ID NO: 33 - protein, 34 aa, synthetic construct
EIDHALPFSRTWDDSFNNKVLVLGSENQNKGNQT

SEQ ID NO: 34 - protein, 34 aa, synthetic construct
DVDHIVPQSFLKDDSIDNKVLTRSDKNRGKSDNV

SEQ ID NO: 35 - protein, 345 aa, synthetic construct
XXXXXXXXXXXXXXXXXXXXXXXXXXXXXXXXXXXXXXXXXXXXXXXXXXXXXXPLVLGIDPGXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXVVLELNRFSXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXHYLDAY

SEQ ID NO: 36 - protein, 345 aa, synthetic construct
XXXXXXXXXXXXXXXXXXXXXXXXXXXXXXXXXXXXXXXXXXXXXXXXXXXXXXPLILGIDPGXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXVVLEVNRFAXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXHHLDAY

SEQ ID NO: 37 - protein, 345 aa, synthetic construct
XXXXXXXXXXXXXXXXXXXXXXXXXXXXXXXXXXXXXXXXXXXXXXXXXXXXXXPLRLKLDPGXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXITQELVRFDXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXHALDAA

SEQ ID NO: 38 - protein, 345 aa, synthetic construct
XXXXXXXXXXXXXXXXXXXXXXXXXXXXXXXXXXXXXXXXXXXXXXXXXXXXXXPLRLKLDPGXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXTILETGSFDXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXHIFDAA

SEQ ID NO: 39 - protein, 345 aa, synthetic construct
XXXXXXXXXXXXXXXXXXXXXXXXXXXXXXXXXXXXXXXXXXXXXXXXXXXXXXNYILGLDIGXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXIHIETAREVXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXHALDAV

SEQ ID NO: 40 - protein, 345 aa, synthetic construct
XXXXXXXXXXXXXXXXXXXXXXXXXXXXXXXXXXXXXXXXXXXXXXXXXXXXXXKYSIGLDIGXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXIVIEMARENXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXHAHDAY

SEQ ID NO: 41 - protein, 4 aa, synthetic construct
GSGS

(19) United States
(12) Patent Application Publication
(10) Pub. No.: US 2025/0283116 A1
(43) Pub. Date: Sep. 11, 2025

(54) USE OF ISCB IN GENOME EDITING

(71) Applicant: Cornell University, Ithaca, NY (US)

(72) Inventors: Ailong KE, Ithaca, NY (US); Gabriel SCHULER, Mt. Airy, MD (US)

(21) Appl. No.: 18/862,660

(22) PCT Filed: May 8, 2023
(86) PCT No.: PCT/US2023/066742

§ 371 Date: Nov. 4, 2024

Related U.S. Application Data
(60) Provisional application No. 63/339,278, filed on May 6, 2022.
(60) Provisional application No. 63/351,301, filed on Jun. 10, 2022.

(51) Int. Cl. C12N 15/90; C12N 9/22; C12N 15/11

(52) U.S. Cl. CPC C12N 15/907; C12N 9/226; C12N 15/11; C12N 2310/20

(57) ABSTRACT

Provided are modified proteins that are functional in RNA-guided DNA cleavage. The proteins include modified IscBs protein that have a modification of the N-terminus or C-terminus, or both. The modifications include a truncation of a PLMP domain of the IscB protein, or a PLMP domain that is relocated to a position of the IscB protein that is not the N-terminus. The modified IscB protein can be provided as a component of a fusion protein. The modified IscB proteins are used with an ωRNA to modify a DNA substrate.

### CROSS-REFERENCE TO RELATED APPLICATIONS

This application claims priority to U.S. provisional patent application No. 63/339,278, filed May 6, 2022, and to U.S. provisional patent application No. 63/351,301, filed Jun. 10, 2022, the entire disclosures of each of which are incorporated herein by reference.

### STATEMENT REGARDING FEDERALLY SPONSORED RESEARCH

This invention was made with government support under grant number R35GM118174 awarded by the National Institutes of Health. The government has certain rights in the invention.

### SEQUENCE LISTING

The instant application contains a Sequence Listing, which is submitted in .xml format is hereby incorporated by reference in its entirety. Said .xml file is named “018617 01416_ST26.xml”, was created on May 8, 2023, and is 46,072 bytes in size.

### RELATED INFORMATION

Increasing evidence points to the possibility that the core components of the CRISPR-Cas adaptive immune systems evolved from genes in mobile genetic elements. The class 2 CRISPR effectors Cas9 (Type II) and Cas12 (Type V) are believed to have independently evolved from an ancestral TnpB-like nuclease, which is still commonly found in insertion sequence (IS) elements today (1-3). Cas9 appears to have emerged from a distinct branch of IS elements within the IS200/605 superfamily harboring IscB (2). IscB and Cas9 share a common domain architecture at the sequence level (FIG. 1A-B). Both contain an arginine-rich bridge helix and an HNH endonuclease domain inserted into a RuvC endonuclease domain (1, 2). The bridge helix in Cas9 plays a crucial role in mediating complex ribonucleoprotein (RNP) formation with two non-coding RNAs, crRNA and tracrRNA (4-8). HNH and RuvC endonucleases are used by Cas9 to cleave the target and nontarget DNA strands, respectively (4). During CRISPR interference, the DNA substrate is validated through R-loop formation, which involves DNA unwinding and RNA/DNA heteroduplex formation (7, 9-14). IscB was found to assemble with a single large (>200 nt) noncoding RNA encoded by the transposon, ωRNA (OMEGA: obligate mobile element guided activity) (2). Together IscB-ωRNA mediates RNA-guided DNA cleavage similar to the Cas9-crRNA-tracrRNA RNP (2). To avoid self-targeting and to reduce search space, Cas9 further specifies a protospacer adjacent motif (PAM) adjacent to the target site (15, 16). This mechanism is conserved in IscB-ωRNA, and the equivalent target-adjacent motif (TAM) is recognized by a TAM interaction domain (TID) in IscB (2). All IscBs further encode a PLMP-motif containing domain at the N-terminus (2). This domain is not found in Cas9 (FIG. 1B). While prior approaches to engineering IsbC proteins for use in DNA modification have been reported, there remains an ongoing and unmet need for improved IscB proteins and systems for DNA modification. The present disclosure is pertinent to this need.

### BRIEF DESCRIPTION OF THE FIGURES

FIGS. 1A-1E. Cryo-EM reconstruction and structure of IscB RNP bound to target DNA. (A) Arrangement of the OgeuIscB and ωRNA in its native IS element defined by the left (LE) and right (RE) ends of the transposon. (B) Domain organization of IscB. P1D, P1 interaction domain; TID, TAM-interaction domain. RuvC domain is separated into three segments: RuvC I, II, and III. Color scheme is conserved throughout FIG. 1. (C) Diagram of R-loop formed between guide RNA and target DNA. TAM sequence is read 5′-CTAGAA-3′ on the non-target strand. (D, E) cryo-EM reconstruction at 2.78 Å and cartoon representations of the IscB-ωRNA/target DNA complex. The sequence in FIG. 1C Guide is top strand is AAAAGAGUGAACGAGA (SEQ ID NO:3); the TAM sequence is TTCTAG; the bottom strand is AAGATCATTTTTTTTGAGAAAA (SEQ ID NO:4).

FIGS. 2A-2D. Structural organization of the ωRNA and comparison to Cas9 crRNA-tracrRNA. (A) Schematic of ωRNA depicting secondary and tertiary interactions. Non-target strand, red; target strand, blue; guide RNA, orange. (B) Atomic model of ωRNA. (C) Close-up view depicting R-loop base pairing between guide RNA and target strand DNA. (D) Structural alignment of ωRNA and tracrRNA-crRNA in SpCas9 RNP showing conserved RNA structures in guide RNAs, P1 with SpCas9 tracrRNA-crRNA helix, J1 with SpCas9 tracrRNA stem loop 1, P3 pseudoknot with SpCas9 tracrRNA stem loop 2, and P5 with SpCas9 tracrRNA stem loop 3. Colored in black is the region of the ωRNA replaced by the REC lobe in Cas9. The sequences on FIG. 2A are:

--GACTAGAAGTCGAGG--
(SEQ ID NO: 26, where “--” corresponds
to an undefined sequence);
--CCTCGACTTCTAGTCTCGTTCACTCTTTT--
(SEQ ID NO: 27, where “--” corresponds
to an undefined sequence);
and
--AAAAGAGTGAACGAGAGGCTCTTCCAACTTNNNNNN
NNNNNNNNNAGGTTGAAAGAGCACAGGCTGAGACATTC
GTAAGGCCGAAGGACCGGACGCACCCTGGGATTTCCCC
AGTCCCCGGAACTGCATAGCGGATGCCAGTTGATNNNN
NNNNNNATCAGATAAGCCAGGGGG
AACAATCACCTCTCTGTATCAGAGAGAGTTTTAC--
(SEQ ID NO: 29, where “--” corresponds
to an undefined sequence)

FIGS. 3A-3F. Structural basis for TAM recognition and R-loop formation by IscB-ωRNA. (A) TAM recognition and R-loop specification by domains of IscB. Color scheme is consistent with FIGS. 1A-1E. (B) Close-up view of P1 interaction domain (P1D) linker residues recognizing TAM-2 basepair (target adjacent motif) from the DNA minor groove side. (C) Close-up view of the IscB TAM interaction domain (TID) making base-specific contacts from the DNA major groove side. (D) Close-up view of the bridge helix and P1D making contacts with the beginning portion of the DNA/RNA heteroduplex in the R-loop region. (E) Close-up view of the β-hairpin+linker domain specifying meandering the minor groove of the middle portion of the DNA/RNA heteroduplex. (F) Diagram of IscB contacts to TAM and DNA/RNA heteroduplex in the R-loop. Positioning of bridge helix domain separating the R-loop from the core of ωRNA in light blue. Green lines denote electrostatic contacts and brown lines denote hydrophobic contacts. TAM highlighted with purple box (ideal TAM sequence: 5′-NWRRNA-3′). guide RNA (orange), target strand DNA (blue), non-target strand DNA (red).

FIGS. 4A-4K. Mechanistic dissection of RNA-guided DNA cleavage by IscB. (A) 3.7 Å EM map and atomic model depicting the unlocked R-loop state. Color scheme is consistent with that in FIGS. 1A-1E. (B) Focused view of DNA, guide RNA, and nuclease densities seen in the unlocked R-loop state. Note that NTS is blocked from entering the RuvC cleavage site by the anchor of HNH to RuvC. (C) 3.8 Å EM map and atomic model of the locked R-loop state. Alphafold predicted HNH domain structure (in green) is docked unambiguously into the EM density. Linker between HNH and RuvC domains can be seen interacting with the TAM-distal portion of the R-loop. (D) Focused view of HNH densities in the locked (active) state. The NTS density is now allowed into the RuvC active site. (E) Close-up view of the HNH active site in the locked state. Catalytic metal ion (black) is seen coordinated to the TS substrate. A second metal ion is required for cleavage (ball with dashed line). It is repelled from the active site by the phosphothioate modification in DNA. (F) Close-up view of the RuvC active site in the locked R-loop state. The coordinated catalytic metal ion (black) is seen contacting the backbone of the incoming NTS DNA which is depicted in cartoon form. (G) Urea-PAGE showing time-resolved DNA cleavage. TS is cleaved by HNH prior to NTS cleavage by RuvC, supporting the unlocked/locked R-loop cleavage model. (H) Proposed mechanistic model explaining ordered strand cleavage by IscB. (I) Small RNA-seq of purified IscB-RNP, showing partial degradation of the guide RNA and a predictable cleavage site preceding stemloop P5. (J) Domain organization of wild-type and ΔPLMP IscB. (K) Urea-PAGE showing time-resolved DNA cleavage by IscB ΔPLMP.

FIGS. 5A-5H. Reconstitution of the IscB-ωRNA RNP. (A) IscB and ωRNA co-expression scheme. (B) Elution profile of the IscB-ωRNA RNP on anion exchange chromatography. (C) Elution profile of IscB-ωRNA RNP on size-exclusion chromatography (SEC). (D) SDS-PAGE analysis of the Strep-tactin purified IscB-ωRNA RNP. Whole cell (WC), lysed pellet (P), lysate supernatant (L), strep resin flow thru (FT), Dnase I wash (W1), wash2 (W2), elution (E). (E) Top: SDS-PAGE of anion exchange peak fractions. Bottom: denaturing-PAGE showing cleavage activity of each fraction. Red channel shows non target strand (NTS). Green channel shows target stand (TS). (F) SDS-PAGE of SEC peak fractions. (G) Denaturing-PAGE showing the ωRNA quality extracted from IscB RNP. Arrows depict the procedural flow of the purification process. Boxes depict the final purified sample in SDS-PAGE gel (protein) and Denaturing-PAGE (RNA). (H) Denaturing urea-PAGE gel showing time-resolved cleavage reaction of cryo-EM sample NTS-DNA containing phosphorothioate bonds. Minimal cleavage of phosphorothioate bonds observed in standard cleavage conditions. The addition of 2 mM MnC12 is shown to rescue cleavage of NTS-DNA by RuvC.

FIGS. 6A-6E. CryoEM single particle reconstruction of IscB-ωRNA-DNA complex. (A, B) Workflow of the cryo-EM image processing and 3D reconstruction for the IscB-ωRNA/DNA complex. Final electron density map with the density from each chain colored separately. (C) Fourier Shell Correlations (FSC) of IscB-ωRNA/DNA complex reconstruction, with the gold-standard cutoff (FSC=0.143) marked with a dotted line. (D) Direction distribution plot. (E) Final electron density map showing local resolution.

FIGS. 7A-7C. Representative local map density for the different functional states. (A) EM densities for representative protein regions inside IscB-ωRNA/DNA complex. (B) EM densities for the target and non-target DNA strands inside the IscB-ωRNA/DNA complex. (C) EM densities for representative RNA regions inside IscB-ωRNA/DNA.

FIGS. 8A-8B. Structural comparison between NmeCas9 RNP and IscB-ωRNA. The NmeCas9 RNP (PDB: 6JDV) is significantly bigger in dimension, fuller in the Z dimension, and makes more extensive contacts with the DNA/RNA heteroduplex in the R-loop region. The lower portion of the R-loop is better protected by the Cas9 RNP, in particular.

FIGS. 9A-9B. Comparative structural analysis of ωRNA and core IscB domains with tracrRNA, guide RNA, and core Cas9 domains (A) Structural comparison between the RNA components of the SpyCas9, NmeCas9, and IscB RNP. All three elements aligned showing structural conservation of ωRNA elements in Cas9 crRNA and tracrRNA. (B) Structural comparison between core protein domains and RNA components of SpyCas9, NmeCas9, and IscB. The bridge helix and RuvC domain are conserved across SpyCas9, NmeCas9, and IscB. All three elements aligned showing structural conservation of the bridge helix, RuvC domains, and ωRNA elements in Cas9 crRNA and tracrRNA.

FIGS. 10A-10E. IscB protein domain interactions with ωRNA and guide RNA. (A) Electrostatic surface representation of IscB superimposed with the cartoon representation of ωRNA. IscB displays extensive positive charges (in blue) on surface for nucleic acid interaction. The bridge helix is boxed in a dashed line. (B) Close-up view of the IscB PLMP domain interactions with the base of P5 in ωRNA. (C) Close-up view of the bridge helix domain making consecutive phosphate backbone contacts to the guide RNA. (D) Close-up view of the IscB β-hairpin+linker domain to the P3 and J2 helices in the ωRNA lobe. (E) Close-up view of P1 interaction domain (P1D) contacting P1 of ωRNA.

FIGS. 11A-11G. Post-refinement to resolve HNH-docked conformational state. (A) Workflow to post-refine the high-resolution IscB-ωRNA/DNA data set. Finer 3D classification to partition HNH-docked conformational state (locked R-loop, 3.1 Å resolution) from the undocked state (unlocked R-loop, 3.2 Å resolution). Out of the 160,000 particles, ˜40,000 exist in the HNH-docked state. (B, C) Local resolution distribution and (D, E) Fourier Shell Correlations of the unlocked and locked R-loop state, respectively. The gold-standard cutoff (FSC=0.143) is marked with a dotted line. (F, G) Direction distribution plot of the unlocked and locked R-loop state, respectively.

FIGS. 12A-12G. Local density for HNH and RuvC domains in locked R-loop (active) state. (A) EM density for HNH domain in locked R-loop state. (B) EM local densities for representative regions in the HNH domain. (C) EM local density of zinc finger in HNH domain. (D) EM local density of HNH active site showing metal ion in black. (E) EM density for RuvC domain in locked R-loop state. (F) EM local densities for representative regions in the RuvC domain. (G) Local EM density of the RuvC active site showing metal ions. Metal ion in black seen in EM density. Metal ion in gray is expected but not seen in density due to phosphorothioate substitution in NTS-DNA.

FIGS. 13A-13D. Comparison of IscB and Cas9 HNH active site. (A) Structural alignment of HINH domain and TS-DNA of SpyCas9 (PDB: 7S4X) and IscB. OgeuIscB, green; OgeuIscB TS-DNA, light blue; SpyCas9, pink; SpyCas9 TS-DNA, blue. (B) Close-up structural alignment of the HNH active site of SpyCas9 and IscB RNP. (C) Amino acid sequence alignment of HNH active site. Triangles mark the Histidine residues coordinating a metal ion in the OgeuIscB structure. Sequence is numbered according to OgeuIscB amino acid sequence. (D) Weblogo of HNH active site of OgeuIscB aligned with top 99 blastp hits in NCBI NR database. The sequences in FIG. 13C are:

(SEQ ID NO: 29)
HYHHVVPRRKNGSETLENRVGLCEEHHRLVHTDK;
(SEQ ID NO: 30)
HYHHVNPRHRNGSETLENRAGLCKEHHFLVHTEE;
(SEQ ID NO: 31)
QIEHIRPKSAGGSNRLSNLTLACAPCNHKKGAQS;
(SEQ ID NO: 32)
EVHHIIFRSRNGSDEEANLLTLCKTCHDGLHAGT;
(SEQ ID NO: 33)
EIDHALPFSRTWDDSFNNKVLVLGSENQNKGNQT;
and
(SEQ ID NO: 34)
DVDHIVPQSFLKDDSIDNKVLTRSDKNRGKSDNV

FIGS. 14A-14B. NTS-DNA in RuvC nuclease center. (A) EM local density of NTS-DNA in locked R-loop (active) structure. NTS-DNA is not seen exiting the nuclease center at high contour level (0.15). (B) EM local density of NTS-DNA in locked R-loop (active) structure at higher contour level (0.071) showing that phosphorothioate bonds in NTS-DNA strand are intact in cryo-EM sample. NTS-DNA is seen exiting nuclease center. NTS-DNA strand in TAM distal R-loop is not observed due to high flexibility.

FIGS. 15A-15D. Comparison of IscB and Cas9 RuvC active site. (A) Structural alignment of the RuvC domain and NTS-DNA of SpyCas9 (PDB: 7S4X) and IscB. OgeuIscB, pink; OgeuIscB NTS-DNA, orange; SpyCas9, light blue; SpyCas9 NTS-DNA, red. (B) Close-up of RuvC active site of SpyCas9 (PDB: 7S4X) and IscB RNP. (C) Amino acid sequence alignment of RuvC active site. Triangles mark the active site residues. Sequence is numbered according to OgeuIscB amino acid sequence. (D) Weblogo of RuvC active site of OgeuIscB aligned with top 99 blastp hits in NCBI NR database. The sequences in FIG. 15C are:

(SEQ ID NO: 35)
XXXXXXXXXX XXXXXXXXXX XXXXXXXXXX XXXXXXXXXX XXXXXXXXXX
XXXXPLVLGI DPGXXXXXXX XXXXXXXXXX XXXXXXXXXX XXXXXXXXXX
XXXXXXXXXX XXXXXXXXXX XXXXXXXXXX XXXXXXXXXX XXXXXXXXXX
XXXXXXXXXX XXXXXXXXXX XXXXXXXXXX XXXXXXXXXV VLELNRFSXX
XXXXXXXXXX XXXXXXXXXX XXXXXXXXXX XXXXXXXXXX XXXXXXXXXX
XXXXXXXXXX XXXXXXXXXX XXXXXXXXXX XXXXXXXXXX XXXXXXXXXX
XXXXXXXXXX XXXXXXXXXX XXXXXXXXXX XXXXXXXXXH YLDAY;
(SEQ ID NO: 36)
XXXXXXXXXX XXXXXXXXXX XXXXXXXXXX XXXXXXXXXX XXXXXXXXXX
XXXXPLILGI DPGXXXXXXX XXXXXXXXXX XXXXXXXXXX XXXXXXXXXX
XXXXXXXXXX XXXXXXXXXX XXXXXXXXXX XXXXXXXXXX XXXXXXXXXX
XXXXXXXXXX XXXXXXXXXX XXXXXXXXXX XXXXXXXXXV VLEVNRFAXX
XXXXXXXXXX XXXXXXXXXX XXXXXXXXXX XXXXXXXXXX XXXXXXXXXX
XXXXXXXXXX XXXXXXXXXX XXXXXXXXXX XXXXXXXXXX XXXXXXXXXX
XXXXXXXXXX XXXXXXXXXX XXXXXXXXXX XXXXXXXXXH HLDAY;
(SEQ ID NO: 37)
XXXXXXXXXX XXXXXXXXXX XXXXXXXXXX XXXXXXXXXX XXXXXXXXXX
XXXXPLRLKL DPGXXXXXXX XXXXXXXXXX XXXXXXXXXX XXXXXXXXXX
XXXXXXXXXX XXXXXXXXXX XXXXXXXXXX XXXXXXXXXX XXXXXXXXXX
XXXXXXXXXX XXXXXXXXXX XXXXXXXXXX XXXXXXXXXI TQELVRFDXX
XXXXXXXXXX XXXXXXXXXX XXXXXXXXXX XXXXXXXXXX XXXXXXXXXX
XXXXXXXXXX XXXXXXXXXX XXXXXXXXXX XXXXXXXXXX XXXXXXXXXX
XXXXXXXXXX XXXXXXXXXX XXXXXXXXXX XXXXXXXXXH ALDAA;
(SEQ ID NO: 38)
XXXXXXXXXX XXXXXXXXXX XXXXXXXXXX XXXXXXXXXX XXXXXXXXXX
XXXXPLRLKL DPGXXXXXXX XXXXXXXXXX XXXXXXXXXX XXXXXXXXXX
XXXXXXXXXX XXXXXXXXXX XXXXXXXXXX XXXXXXXXXX XXXXXXXXXX
XXXXXXXXXX XXXXXXXXXX XXXXXXXXXX XXXXXXXXXT ILETGSFDXX
XXXXXXXXXX XXXXXXXXXX XXXXXXXXXX XXXXXXXXXT XXXXXXXXXX
XXXXXXXXXX XXXXXXXXXX XXXXXXXXXX XXXXXXXXXX XXXXXXXXXX
XXXXXXXXXX XXXXXXXXXX XXXXXXXXXX XXXXXXXXXH IFDAA;
(SEQ ID NO: 39)
XXXXXXXXXX XXXXXXXXXX XXXXXXXXXX XXXXXXXXXX XXXXXXXXXX
XXXXNYILGL DIGXXXXXXX XXXXXXXXXX XXXXXXXXXX XXXXXXXXXX
XXXXXXXXXX XXXXXXXXXX XXXXXXXXXX XXXXXXXXXX XXXXXXXXXX
XXXXXXXXXX XXXXXXXXXX XXXXXXXXXX XXXXXXXXXI HIETAREVXX
XXXXXXXXXX XXXXXXXXXX XXXXXXXXXX XXXXXXXXXX XXXXXXXXXX
XXXXXXXXXX XXXXXXXXXX XXXXXXXXXX XXXXXXXXXX XXXXXXXXXX
XXXXXXXXXX XXXXXXXXXX XXXXXXXXXX XXXXXXXXXH ALDAV;
and
(SEQ ID NO: 40)
XXXXXXXXXX XXXXXXXXXX XXXXXXXXXX XXXXXXXXXX XXXXXXXXXX
XXXXKYSIGL DIGXXXXXXX XXXXXXXXXX XXXXXXXXXX XXXXXXXXXX
XXXXXXXXXX XXXXXXXXXX XXXXXXXXXX XXXXXXXXXX XXXXXXXXXX
XXXXXXXXXX XXXXXXXXXX XXXXXXXXXX XXXXXXXXXI VIEMARENXX
XXXXXXXXXX XXXXXXXXXX XXXXXXXXXX XXXXXXXXXX XXXXXXXXXX
XXXXXXXXXX XXXXXXXXXX XXXXXXXXXX XXXXXXXXXX XXXXXXXXXX
XXXXXXXXXX XXXXXXXXXX XXXXXXXXXX XXXXXXXXXH AHDAY.

FIGS. 16A-16C. PLMP mutant cleavage. (A) Cyro-EM reconstruction of IscB highlighting PLMP domain (green) and P5 (red). (B) Denaturing urea-PAGE cleavage gel showing RNA guided DNA cleavage with IscB ΔPLMP. T (target dsDNA), NT (non-target dsDNA control). (C) Time resolved cleavage of wt IscB compared with ΔPLMP IscB.

FIG. 17 shows a cartoon depiction of a modified IscB protein where the PLMP domain has been moved to the C-terminus, separated by a GS linker.

FIG. 18 provides a photographic depiction of an SDS PAGE gel demonstrating production of the modified IscB protein depicted in FIG. 17. Annotations on the figure are as follows: PLMP PE: OgeuIscB with PLMP mutation and prime editing ωRNA (with 5′ wrRNA guide exonuclease protection and without P5 stem loop); opt: optimized OgeuIscB; circ: circular permutation OgeuIscB; lysate: cell lysate after sonication and centrifugation; FT: strep tactin flow through; W: strep tactin resin wash; E: strep tactin resin elution with 3C protease cleavage.

FIG. 19 provides a photographic representation of a urea gel, obtained using the modified IscB protein as shown in FIG. 17 and produced as described in FIG. 18 in a prime editing experiment.

FIG. 20 provides additional data using the modified IscB protein in prime editing experiments.

### SUMMARY

The present disclosure provides modified IscB proteins that in some embodiments are functional in RNA-guided DNA cleavage. The modified IscB proteins have in some embodiments a modification of an N-terminus or C-terminus, or both. The modifications include a truncation of a PLMP domain of the IscB protein, or a PLMP domain that is relocated to a position of the IscB protein that is not N-terminus, including but not necessarily limited to the C-terminus. The modified IscB proteins may comprise mutations that impart improved properties the modified proteins. The improved properties include but are not necessarily limited to increased polynucleotide binding and/or editing activity, relative to an unmodified IscB protein. The IscB proteins can be provided as a component of fusion proteins to enhance or alter function, or gain a new function. The modified IscB proteins are used with an ωRNA to bind to a polynucleotide substrate and may edit the polynucleotide substrate. The disclosure includes introducing into cells a modified IscB protein and an ωRNA. The modified IscB protein and the ωRNA bind to a target polynucleotide in a ωRNA-guided manner. The IscB protein and the ωRNA may modify the target to, for example, create an indel. The disclosure includes cDNAs and expression vectors, such as viral expression vectors, that express the modified IscB protein and may also express the ωRNA.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

As used in the specification and the appended claims, the singular forms “a” “and” and “the” include plural referents unless the context clearly dictates otherwise. Ranges and other values may be expressed herein as from “about” or “approximately” one particular value, and/or to “about” or “approximately” another particular value. When values are expressed as approximations by the use of the antecedent “about” or “approximately” it will be understood that the particular value forms another embodiment. The term “about” and “approximately” in relation to a numerical value encompasses variations of +/−10%, to +/−1%.

The disclosure includes all steps and reagents such as proteins and nucleic acids, and all combinations of steps reagents, described herein, and as depicted on the accompanying figures. The described steps may be performed as described, including but not necessarily sequentially.

The disclosure includes all polynucleotide and amino acid sequences described herein. Each RNA sequence includes its DNA equivalent, and each DNA sequence includes its RNA equivalent. Complementary and anti-parallel polynucleotide sequences are included. Every DNA and RNA sequence encoding polypeptides disclosed herein is encompassed by this disclosure. Amino acids of all protein sequences and all polynucleotide sequences encoding them are also included, including but not limited to sequences included by way of sequence alignments. Sequences of from 80.00%-99.99% identical to any sequence (amino acids and nucleotide sequences) of this disclosure are included. The disclosure includes any protein having at least 80% amino acid sequence identity with a specific amino acid sequence defined herein by way of a sequence identifier or database entry. Percent amino acid sequence identity with respect proteins means the percentage of amino acid residues in another sequence that are identical with the amino acid residues in the defined sequence, after aligning the sequences in the same reading frame and introducing gaps, if necessary, to achieve the maximum percent sequence identity, and optionally not considering any conservative substitutions as part of the sequence identity.

The disclosure includes all polynucleotide and all amino acid sequences that are identified herein by way of a database entry. Such sequences are incorporated herein as they exist in the database on the filing date of this application or patent.

Amino-acid residue sequences described herein are represented herein by formulae whose left and right orientation is in the conventional direction of amino-terminus to carboxy-terminus, unless stated differently. Additionally, a dash at the beginning or end of an amino acid sequence may indicate a peptide bond to a further sequence comprising one or more amino-acid residues.

The disclosure includes all amino acid sequences that are defined by sequence identifier, but with one or more changed amino acids, relative to a native amino acid sequence. Amino acid changes include conservative changes, such as by changing an amino acid belonging to a grouping of amino acids having a particular size or characteristic to an amino acid belonging to the same grouping, and non-conservative changes, such as such as changing an amino acid belonging to a grouping of amino acids having a particular size or characteristic to an amino acid belonging to another grouping.

In embodiments the disclosure provides functional IscB proteins, and methods and systems that use the functional IscB protein. The described IscB proteins may be modified relative to their unmodified versions. A functional modified IscB protein is a modified IscB protein that can cleave DNA in an RNA guided system. As such, the disclosure provides unexpectedly functional IscB proteins in view of the disclosure of PCT publication WO/2022/087494, which discloses that the IscB PLMP domain is essential for RNA-guided cleavage function, and that truncations of a segment of an IscB protein that comprises a PLMP domain abolishes activity. In contrast to this description in PCT publication WO/2022/087494, the present disclosure demonstrates that the IscB domain can be removed from the full length IscB protein to provide a truncated IscB protein, but the truncated IscB protein retains DNA cleavage activity. PCT WO/2022/087494 also describes the position of the PLMP domain at the N-terminus of the IscB protein. In contrast, the present specification also demonstrates that the PLMP domain can be repositioned away from its native N-terminal position, yet the modified IscB protein retains DNA cleavage activity. The present specification also includes IscB proteins with truncated or repositioned PLMP domains, but wherein the IscB protein may be rendered catalytically inactive, e.g., a nuclease dead IscB protein. Thus, in embodiments, the disclosure provides an IscB protein that comprise a truncation of amino acids from its N-terminal end. In embodiments, the disclosure provides an IscB protein comprising an N-terminal truncation, and wherein optionally the truncation comprises at least 30, 35, 40, 45, 50, 55, 60, 65, or 70 amino acids. In an embodiment, the disclosure provides an IscB protein comprising an N-terminal truncation, and wherein optionally the truncation comprises truncation of a PLMP domain, a P5 binding domain, or a combination thereof. In an embodiment, the disclosure provides an IscB protein comprising a truncation and wherein the truncated IscB protein comprises fewer than 450 amino acids. In embodiments, the disclosure provides an isolated or recombinantly produced IscB protein comprising a gut microbiome derived OgeuIscB.

The disclosure provides modified IscB proteins that have amino acid changes, relative to naturally occurring IscB protein. Representative amino acid changes are provided in Table A. The described IscB protein can comprise only one of the described mutations, or any combination thereof. In embodiments, the described IscB protein comprises a combination of 2, 3, 4, 5, 5, 6, 7, 8, 9, 10, 11, 12, 13 or 14 amino acids changes relative to a native IscB amino acid sequence, wherein the amino acid changes are optionally selected from those listed in Table A. The mutations described in Table A are numbered according to a wild type (e.g., a native) IscB protein that has the sequence:

(SEQ ID NO: 1)
MAVVYVISKSGKPLMPTTRCGHVRILLKEGKARVVERKPFTIQLT
YESAEETQPLVLGIDPGRTNIGMSVVTESGESVFNAQIETRNKDV
PKLMKDRKQYRMAHRRLKRRCKRRRRAKAAGTAFEEGEKQRLLPG
CFKPITCKSIRNKEARFNNRKRPVGWLTPTANHLLVTHLNVVKKV
QKILPVAKVVLELNRFSFMAMNNPKVQRWQYQRGPLYGKGSVEEA
VSMQQDGHCLFCKHGIDHYHHVVPRRKNGSETLENRVGLCEEHHR
LVHTDKEWEANLASKKSGMNKKYHALSVLNQIIPYLADQLADMFP
GNFCVTSGQDTYLFREEHGIPKDHYLDAYCIACSALTDAKKVSSP
KGRPYMVHQFRRHDRQACHKANLNRSYYMGGKLVATNRHKAMDQK
TDSLEEYRAAHSAADVSKLTVKHPSAQYKDMSRIMPGSILVSGEG
KLFTLSRSEGRNKGQVNYFVSTEGIKYWARKCQYLRNNGGLQIYV
GS.

TABLE A

| | Average editing | Standard |
|---|---|---|
| | efficiency from | deviation |
| IscB mutation | triplicates (%) | (± %) |
| Non-targeting control (no RNP) | 0.00 | 0.0 |
| Wild-type IscB | 0 | 0 |
| T64R | 0.00 | 0.0 |
| N65R | 0.00 | 0.0 |
| K88R | 1.50 | 1.3 |
| K95R | 1.43 | 0.9 |
| D89R | 0.22 | 0.2 |
| N65R | 0.00 | 0.0 |
| P91T | 0.00 | 0.0 |
| P91R | 0.00 | 0.0 |
| M102R | 2.57 | 1.4 |
| K138R | 0.23 | 0.4 |
| F152R | 0.00 | 0.0 |
| N154R | 0.00 | 0.0 |
| K156R | 1.48 | 1.0 |
| T163R | 0.00 | 0.0 |
| T165R | 0.00 | 0.0 |
| Q212R | 0.18 | 0.3 |
| K291R | 0.00 | 0.0 |
| V298R | 0.00 | 0.0 |
| N300R | 0.00 | 0.0 |
| Y305R | 0.00 | 0.0 |
| K337R | 0.00 | 0.0 |
| H339R | 0.00 | 0.0 |
| K392R | 1.45 | 1.0 |
| L393K | 0.07 | 0.1 |
| M402R | 0.00 | 0.0 |
| D403R | 0.00 | 0.0 |
| K405R | 0.00 | 0.0 |
| T406R | 0.89 | 1.1 |
| K427R | 0.70 | 0.2 |
| S430R | 1.65 | 1.1 |
| Y468R | 0.00 | 0.0 |
| K476R | 1.39 | 1.1 |
| W478R | 0.00 | 0.0 |
| K481R | 1.54 | 0.7 |

Table A also provides a summary of results using a described IscB system with a guide RNA targeting a VEGF site. Increased editing efficiency of described mutants as determined by indel production averaged over triplicate experiments is provided. Thus, in non-limiting examples the disclosure provides modified IscB proteins that exhibit gain of function, when used in combination with a targeting RNA as further described herein. The gain of function may comprise increased DNA editing relative to an unmodified IscB protein used with the same targeting RNA. In non-limiting embodiments, the disclosure provides modified IscB protein that comprise at least one mutation selected from K88R, K95R, D89R, 10 M102R, K138R, K156R, Q212R, K392R, L393K, T406R, K427R, S430R, K476R, and K481R. The relative positions of these mutations and other mutations described herein in the context of an IscB protein that has its PLMP domain removed or reposition will be understood by those skilled in the art by accounting for removed or repositioned PLMP domain amino acids by subtracting 54 (i.e., the 54 amino acids of the PLMP domain) from the N-terminus for a truncation, and adding the 54 amino acids of the PLMP domain for a repositioning to the C-terminus, by comparison to SEQ ID NO:1, e.g., an intact IscB protein sequence. Thus, the disclosure includes each mutation described in Table A, where the location of the mutation is the stated number, minus 54. In one embodiment, an IscB protein that has had the PLMP domain removed has the sequence:

(SEQ ID NO: 2)
LVLGIDPGRTNIGMSVVTESGESVFNAQIETRNKDVPKLMKDRKQ
YRMAHRRLKRRCKRRRRAKAAGTAFEEGEKQRLLPGCFKPITCKS
IRNKEARFNNRKRPVGWLTPTANHLLVTHLNVVKKVQKILPVAKV
VLELNRFSFMAMNNPKVQRWQYQRGPLYGKGSVEEAVSMQQDGHC
LFCKHGIDHYHHVVPRRKNGSETLENRVGLCEEHHRLVHTDKEWE
ANLASKKSGMNKKYHALSVLNQIIPYLADQLADMFPGNFCVTSGQ
DTYLFREEHGIPKDHYLDAYCIACSALTDAKKVSSPKGRPYMVHQ
FRRHDRQACHKANLNRSYYMGGKLVATNRHKAMDQKTDSLEEYRA
AHSAADVSKLTVKHPSAQYKDMSRIMPGSILVSGEGKLFTLSRSE
GRNKGQVNYFVSTEGIKYWARKCQYLRNNGGLQIYVGS.

This sequence may be referred to herein as a IscB ΔPLMP.

In addition to the mutations of Table A, a modified IscB protein may comprises additional amino acid changes, such as Cysteine to Serine mutations which may be located at any of amino acid positions 21, 112, 320, and 379 of SEQ ID NO:1. In this regard, mutants of Ogeu IscB protein were generated and tested. Cysteine to Serine mutations at positions 111, 319, and 378 (Cys111Ser, Cys319Ser, and Cys378Ser) were generated in a single IscB protein. The mutant IscB proteins possessed a number of advantages, including but not limited to improved yield upon overexpression and/or improved stability.

As discussed herein, in an embodiment, the disclosure provides a functional modified IscB protein comprising a removed or repositioned PLMP domain. In an embodiment, the PLMP domain comprises or consists of the sequence: MAVVYVISKSGKPLMPTTRCGHVRILLKEGKARVVERKPFTIQLTYESAEETQP (SEQ ID NO:5) or a sequence having at least 80% identity with the described sequence. In embodiments, an IscB protein of this disclosure includes the sequence of SEQ ID NO:1, but without the PLMP domain of SEQ ID NO:5.

As described above, a full length IscB protein may comprise SEQ ID NO:1. In embodiments, the full length IscB comprises segments, which can be considered domains, as follows:

(SEQ ID NO: 1)
MAVVYVISKSGKPLMPTTRCGHVRILLKEGKARVVERKPFTIQLT
YESAEETQPLVLGIDPGRTNIGMSVVTESGESVENAQIET_{RNKDV}
_{PKLMKDRKQYRMAHRRLKRRCKRRRRAKAA}GTAFEEGEKQRLLPG
CFKPITCKSIRNKEARFNNRKRPVGWLTPTANHLLVTHLNVVKKV
QKILPVAKVVLELNRFSFMAMNNPK^{VQRWQYQRGPLYGKGSVEE}
^{AVSMQQDGHCLFCKHGIDHYHHVVPRRKNGSETLENRVGLCEEHH}
^{RLVHTDKEWEANLASKKSGMINKKYH}ALSVLNQIIPYLADQLAD
MFPGNFCVTSGQDTYLFREEHGIPKDHYLDAYCIACSALTDAKKV
SSPKGRPYMVHQFRRHD [symbol] [symbol] nrsyymggklvatnrhkam
dqktdsleeyraahsaadvskltvkhp [symbol] [symbol] [symbol] [symbol] [symbol]
RNNGGLQ
IYVGS.

As shown, and without intending to be constrained by any particular interpretation, it is considered that the bold amino acids are the PLMP domain; italicized amino acids are RuvC domains; subscripted amino acids are a bridge helix domain; superscripted amino acids are an HINH domain; lowercase amino acids form a domain that makes structure-specific interactions with P1 of ωRNA; enlarged amino acids are a TAM (Target Adjacent Motif). The disclosure includes modifying any one or combination of these domains with amino acid substitutions, insertions, and deletions.

In addition to removal of the IscB PLMP domain, the disclosure includes repositioning the PLMP domain such that it is in a location that is different from its location in the unmodified protein. In an embodiment, the PLMP domain is moved to the C-terminus of the PLMP protein.

A representative sequence of a modified IscB protein having the PLMP domain moved to the C-terminus is:

(SEQ ID NO: 6)
PLVLGIDPGRTNIGMSVVTESGESVFNAQIETRNKDVPKLMKDRK
QYRMAHRRLKRRCKRRRRAKAAGTAFEEGEKQRLLPGCFKPITCK
SIRNKEARFNNRKRPVGWLTPTANHLLVTHLNVVKKVQKILPVAK
VVLELNRFSFMAMNNPKVQRWQYQRGPLYGKGSVEEAVSMQQDGH
CLFCKHGIDHYHHVVPRRKNGSETLENRVGLCEEHHRLVHTDKEW
EANLASKKSGMNKKYHALSVLNQIIPYLADQLADMFPGNFCVTSG
QDTYLFREEHGIPKDHYLDAYCIACSALTDAKKVSSPKGRPYMVH
QFRRHDRQACHKANLNRSYYMGGKLVATNRHKAMDQKTDSLEEYR
AAHSAADVSKLTVKHPSAQYKDMSRIMPGSILVSGEGKLFTLSRS
EGRNKGQVNYFVSTEGIKYWARKCQYLRNNGGLQIYVMAVVYVIS
KSGKPLMPTTRCGHVRILLKEGKARVVERKPFTIQLTYESAEETQ.

An amino acid linker can be included between the repositioned PLMP domain and the remainder of the IscB sequence. In an embodiment, any amino acid linker of this disclosure may comprise comprises Gly and Ser amino acids. In an embodiment the linker begins in the position immediately after amino acid 444 in SEQ ID NO:6. In an embodiment, the linker comprises at least three amino acids. In an embodiment, the linker may be lengthened compared to standard linker lengths to, for example, permit more accessibility for an N-terminal nuclear localization signal (NLS). In a non-limiting embodiment the linker comprises the sequence GGGGSGGGGSGGGGS (SEQ ID NO:7). Thus, any linker used in connection with an IscB protein of this disclosure may comprises at least 3 amino acids. In embodiments, the linker is more than 3 amino acids. In embodiments, the linker is 3-20 amino acids.

An IscB protein comprising a PLMP domain relocated to the N-terminus was analyzed. A cartoon depiction of the modified PLMP is provided in FIG. 17. The sequence of the modified PLMP that was tested is:

(SEQ ID NO: 8)
MGWSHPQFEKGGGSGGGSGGSAWSHPQFEKSDLEVLFQGPLGSPK
KKRKVGS^{KRPAATKKAGQAKKKK}GS_{PKKKRKV}GGGGSPLVLGIDP
GRTNIGMSVVTESGESVFNAQIETRNKDVPKLMKDRKQYRMAHRR
LKRRCKRRRRAKAAGTAFEEGEKQRLLPGCFKPITCKSIRNKEAR
FNNRKRPVGWLTPTANHLLVTHLNVVKKVQKILPVAKVVLELNRF
SFMAMNNPKVQRWQYQRGPLYGKGSVEEAVSMQQDGHCLFCKHGI
DHYHHVVPRRKNGSETLENRVGLCEEHHRLVHTDKEWEANLASKK
SGMNKKYHALSVLNQIIPYLADQLADMFPGNFCVTSGQDTYLFRE
EHGIPKDHYLDAYCIACSALTDAKKVSSPKGRPYMVHQFRRHDRQ
ACHKANLNRSYYMGGKLVATNRHKAMDQKTDSLEEYRAAHSAADV
SKLTVKHPSAQYKDMSRIMPGSILVSGEGKLFTLSRSEGRNKGQV
NYFVSTEGIKYWARKCQYLRNNGGLQIYVGGGGSGGGGSGGGGSG
GGGMAVVYVISKSGKPLMPTTRCGHVRILLKEGKARVVERKPFTI
QLTYESAEETQ.

The sequence in bold is a twin-strep tag. The sequence in italics is an HRV protease cleavage site. The superscripted sequence is a nucleoplasm NLA. The subscripted sequence is an SV40 NLS. A linker sequence is enlarged. The sequence following the linker is the repositioned PLMP domain. Results demonstrating production of this modified IscB protein are shown in FIG. 18. “Circular permutation” refers to repositioning of the PLMP domain. The protein is not circularized. FIG. 19 shows photographic results obtained using the modified IscB protein in a prime editing experiment. “Circular” refers to the IscB construct with the repositioned PLMP domain. The image is of a urea-PAGE denaturing gel showing RNA guided cleavage activity by the IscB constructs. Introducing a template region into the ωRNA allowed for reconstitution of the prime editing activity in vitro. In this example the reverse transcriptase is provided in trans.

As further described herein, the described proteins can be provided in systems that include the described proteins and a guide RNA, referred to herein as a ωRNA and omega RNA. The ωRNA can be provided as a single RNA polynucleotide or may be split into two RNA polynucleotides. A representative single omega guide RNA is:

(SEQ ID NO: 9)
NNNNNNNNNNNNNNNNGGCUCUUCCAACUUUAUGGUUGCGACCGUA
GGUUGAAAGAGCACAGGCUGAGACAUUCGUAAGGCCGAAAGACCG
GACGCACCCUGGGAUUUCCCCAGUCCCCGGAACUGCAUAGCGGAU
GCCAGUUGAUGGAGCAAUCUAUCAGAUAAGCCAGGGGGAACAAUC
ACCUCUCUGUAUCAGAGAGAGUUUUACAAAAGGAGGAACGG

where the italicized Ns target a spacer in a DNA substrate. In an embodiment where the omega RNA is split, representative sequence are:

(SEQ ID NO: 10)
5′-NNNNNNNNNNNNNNNNGGCUCUUCCAACUUUAUGGU,
and
(SEQ ID NO: 11)
5′-UGCGACCGUAGGUUGAAAGAGCACAGGCUGAGACAUUCGUAA
GGCCGAAAGACCGGACGCACCCUGGGAUUUCCCCAGUCCCCGGAA
CUGCAUAGCGGAUGCCAGUUGAUGGAGCAAUCUAUCAGAUAAGCC
AGGGGGAACAAUCACCUCUCUGUAUCAGAGAGAGUUUUACAAAAG
GAGGAACGG.

In a non-limiting embodiment the ωRNA comprises a nucleotide sequence having 80%, 85%, 90%, 95%, or 97% sequence identity to:

(SEQ ID NO: 12)
AAAAGAGUGAACGAGAGGCUCUUCCAACUUUAUGGUUGCGACCGU
AGGUUGAAAGAGCACAGGCUGAGACAUUCGUAAGGCCGAAAGACC
GGACGCACCCUGGGAUUUCCCCAGUCCCCGGAACUGCAUAGCGGA
UGCCAGUUGAUGGAGCAAUCUAUCAGAUAAGCCAGGGGGAACAAU
CACCUCUCUGUAUCAGAGAGAGUUUUACAAAAGGAGGAACGG.

FIG. 20 provides additional prime editing results obtained using the modified IscB protein depicted in FIG. 17. In FIG. 20, the DNA target has 5′ 6-FAM (fluorescein) modification on the non-target strand. Lane 1 shows a ssDNA ladder; lane 2 shows target DNA; lane 3 shows cleavage of target DNA using wt OgeuIscB; lane 4 shows cleavage of target DNA using OgeuIscB ΔPLMP with Prime editing ωRNA; lane 5 shows reverse transcriptase activity extending cleaved non-target strand using 3′ ωRNA template; lane 6 shows reverse transcriptase activity is abolished without the presence of dNTPs,

A prime editing ωRNA DNA coding sequence used in the prime editing figures is:

(SEQ ID NO: 13)
CGCCCCATCAAAAAAATATTgaCAACATAAAAAACTTTGTGTAAT
ACTTGTAACGCTGGGUG^{UCAGGCCUGCUAGUCAGCCACAGCUUGG}
^{GGAAAGCUGUGCAGCCUGUGACCCCCCCAGGAGAAGCUGGG}aatt
ATCAaaaagagtgaacgaga_{GgctcttTcaacttGAAAaggtt}
_{gaaagagcacaggctgagacattcgtaaggccgaaagGccggacg}
_{caccctgggatttccccagtccccggaactgcatagcggatgtca}
_{gttgat}CGGCCGAGTAATTTACGTCGACGTTGACGTCGATGGTTG
CGGCCGatcagataagccagggggaacaatctttcagacttGGAT
CC [symbol] [symbol] AAAGCTAAGG
ATTTTTTTT.

In this DNA coding sequence, the bold nucleotides are an lpp promoter; the superscripted nucleotides are an xeRNA for 5′ exonuclease protection; the unchanged font nucleotides beginning with CGG are a sephadex aptamter; the italicized nucleotides are the ωRNA guide sequence; the subscripted nucleotides are an optimized ωRNA without a P5 stemloop; the bold and italicized nucleotides are an RT template; the enlarged nucleotides are the RT primer binding site; that is followed by decreased font nucleotides which are a transcription terminator.

An example of an optimized ωRNA DNA coding sequence is:

(SEQ ID NO: 14)
CGCCCCATCAAAAAAATATTgaCAACATAAAAAACTTTGTGTAATA
CTTGTAACGCTGaaaagagtgaacgagaggctcttTcaacttGAA
AaggttgaaagagcacaggctgagacattcgtaaggccgaaagGc
cggacgcaccctgggatttccccagtccccggaactgcatagcgg
atgtcagttgatCGGCCGAGTAATTTACGTCGACGTTGACGTCGA
TGGTTGCGGCCGatcagataagccagggggaacaatcacctctct
gGAAAcagagagagttttttttATCCTTAGCGAAAGCTAAGGATT
TTTTTT.

In this sequence, in the bold font nucleotides capitalized nucleotides are mutations to correct mismatches; GAAA is a tetraloop added to P1 and P2 to improve folding, and a series of Us are after P5 to increase termination. Ribonucleoproteins comprising ωRNA encoded by the above construct have been produced and show that the ωRNA produced is resistant to degradation whereas non-optimized RNA is degraded. The disclosure includes use of affinity-purification handles that are engineered into the RNA sequence without affecting the dsDNA cleavage activity of IscB-ωRNA. Such a handle may be replaced with RNA aptamer sequences for fluorescent tagging, chromatine binding (by binding to HnRNP, spliceosome components, and the like), and recruiting protein factors for chromatin modifications in combination with a nuclease dead-IscB protein.

IscB proteins of this disclosure can be provided as fusion proteins which may include any suitable linker, non-limiting examples of which are described herein. Additional amino acids can be added to the N-terminus, the C-terminus, or both, of any IscB protein described herein. In one embodiment a fusion protein of the disclosure includes a described IscB protein segment and a distinct protein segment. A distinct protein segment means a protein or segment of a fusion protein that is not the IscB protein sequence. In embodiments, any IscB protein described herein may be provided as a component of a fusion protein that further comprises a protein segment that is capable of influencing interaction of the fusion protein with nucleic acids. In embodiments, the fusion protein comprises an IscB protein segment at the N-terminus and additional amino acids at the C-terminus. In embodiments, the additional amino acids are an enzyme, or a non-enzymatic nucleic interaction domain. In embodiments, a DNA or RNA binding domain is included in the fusion protein. In embodiments, a domain that is capable of activating or inhibiting transcription, such as for use in CRISPR-i and CRISPR-a applications. In embodiments, a domain that interacts with single or double stranded DNA (i.e., a nucleic acid interaction domain) can be included in a fusion protein. In embodiments, a domain that interacts with a nucleosome can be included in a fusion protein. In embodiments, a domain that interacts with RNA can be included. A non-limiting example of an RNA binding domain is a lambda protein, such as a lambdaN peptide, the sequence of which is known in the art. Another RNA binding domain is the phage derived P22 binding domain, as described further below.

With an RNA binding domain, the disclosure includes use of an RNA that comprises and RNA binding domain binding sequence. In an embodiment, an RNA binding domain is present in an omega RNA, and configured so that the RNA interacting domain improves assembly of the IscB and omega RNA in vivo, such as in a ribonucleoprotein, which may improve editing efficiency.

In embodiments, a described fusion protein can comprise any suitable nuclear localization signal, an organelle targeting signal, a polymerase, a ligase, a helicase, a topoisomerase, or a nucleotide modifying enzyme. Thus, in embodiments, the fusion protein can comprise the IscB segment and segment with enzymatic activity. As a non-limiting example, the fusion protein may comprise a segment that has reverse transcriptase activity. As such, the disclosure provides a fusion protein comprising a described IscB protein segment and a reverse transcriptase (RT) to, for example, facilitate prime editing. In the case of an RT as a component of a fusion protein, non-limiting examples of suitable RTs include M-MLV RT, Marathon RT and GsI-IIC RT. In the RT-fusion approach, in one embodiment, the disclosure provides for addition of a multifunctional prime editing guide RNA (pegRNA). Any protein component of a described fusion protein may also be provided in trans, i.e., a combination of the IscB protein and a separate protein may be provided and used in the described methods.

Data obtained using RNP administration of IscB proteins and guide RNA are presented in Table 2. The data represent RNP-based ex vivo genome editing data in human HEK293 cells targeting the VGFA gene.

In order to produce the data shown in Table B, RNPs were reconstituted and purified from E. coli cell and electroporated into human HEK293 cells. Cells were harvested 72 hours after RNP delivery. A ˜250 bp region around the VGFA target site as PCR-amplified from the genomic DNA of each editing experiment and subjected to Illumina-based deep sequencing. Indels were identified using the Program CRISPResso2.

Each editing experiment was carried out in biological triplicates. Editing efficiencies were calculated from Mean indel frequency. The standard deviations indicate the editing data were consistent and reproducible, taking into account that the sequencing results reported a 0.05% indel frequency from the unedited cells. The observed indels in the unedited cells were all single or less frequently double nucleotide deletions, and therefore may be due to sequencing errors. The indel patterns in the RNP-edited cells are very different and the majority are three nucleotides and longer, and are therefore considered to be true indels.

TABLE B

| | Mean Indel | Standard |
|---|---|---|
| Name of the IscB RNP delivered into the | from | deviation |
| HEK293 cells | triplicates (%) | (%) |
| Unedited HEK293 cells | 0.05 | 0.01 |
| RNP containing wild-type IscB protein | 0.09 | 0.02 |
| RNP containing permutated IscB | 2.23 | 0.09 |
| RNP containing ΔPLMP | 0.06 | 0.02 |
| RNP containing ΔPLMP, C-ter P22 tail, and | 0.08 | 0.02 |
| additional mutations | | |

A P22 peptide sequence (GNAKTRRHERRRKLAIERDTIGY (SEQ ID NO:15)) was fused to the C-terminus of IscB/ΔPLMP, through a 4-AA GSGS (SEQ ID NO:41) linker. Additional mutations were introduced into the protein.

The guide RNA sequence used in the RNP delivery experiment is as follows (provided as DNA sequence, wherein the RNA sequence replaces T's with U's):

(SEQ ID NO: 16)
aaaagagtgaacgagaggctcttTcaacttGAAAaggttgaaaga
gcacaggctgagacattcgtaaggccgaaagGccggacgcaccct
gggatttccccagtccccggaactgcatagcggatgTcagttgat
CGGCCGAGTAATTTACGTCGACGTTGACGTCGATGGTTGCGGCCG
atcagataagccagggggaacaatcacctctctgGAAAcagagag
agtttttttt

The lowercase italicized sequence is a 16 nucleotide segment that targets the VGFA gene. The uppercase T following the 16 nucleotide segment is and the lone downstream uppercase T are nucleotide substitutions to introduce a Watson-Crick base-pair in P1 of Omega RNA. The first uppercase GAAA and a downstream gaaa shortens P1, and introduces a GAAA tetraloop to stabilize the P1 stemloop. The lone uppercase G is a nucleotide substitution to introduce a Watson-Crick pairing in P4, to stabilize the stemloop. The uppercase italicized nucleotides are an introduced a Sephadex aptamer domain in the P4 domain. This is not related to editing, but to improve RNP purification and is optional because it can be replaced with a GAAA tetraloop. The second uppercase GAAA is a tetraloop to stabilize P5.

In any embodiment of the disclosure, a DNA repair template may be used, but the disclosure includes the proviso that the described IscB systems may be used in a DNA repair template-free manner. Where a DNA repair template is used in can include a cargo sequence and if desired left and right homology arms. The cargo sequence may encode a protein or a functional polynucleotide, such as a functional RNA.

In any fusion protein of this disclosure, the protein that is added to the IscB protein to construct the fusion protein may be substituted for the PLMP domain, or be added to the N- or C-terminus of an intact, truncated, or rearranged IscB protein. In embodiments, a fusion protein comprises additional amino acids that are added to a described IscB protein. In embodiments, additional amino acids include any one or a combination of a protein purification tag, such as a Sumo or histidine tag, one or more nuclear localization signals (NLS), ribosomal skipping sequences, protease recognition sequences, and linker sequences. Non-limiting embodiments embodiment of a nuclear localization signal sequence comprises a nucleoplasm NLS having the sequence KRPAATKKAGQAKKKK (SEQ ID NO:17) and SV40 NLS having the sequence PKKKRKV (SEQ ID NO:18). Other NLS signals can be used and, in general, for eukaryotic purposes, a nuclear localization signal comprises one or more short sequences of positively charged lysines or arginines.

In an embodiment, an IscB protein may be rendered catalytically inactive by, for example, introducing point mutations in the HNH domain to inactivate nuclease activity on the target strand.

In embodiments, use of an IscB protein described herein in conjunction with a suitable omega RNA binds to and optionally modifies an polynucleotide substrate. One or more IscB proteins and one or more omega RNA can be used. In embodiments, only one strand of DNA is nicked. In embodiments, both strands of a double stranded DNA molecule are nicked. In embodiments both strands of a double stranded DNA are nicked. Thus, by using a described system, a double stranded DNA break may be produced.

In embodiments, a described system comprising an IscB protein and an omega RNA is used for producing an indel, which may be achieved in a DNA repair template free manner. In embodiments, the indel corrects a mutation in an open reading frame encoded by a selected chromosome locus or converts a sequence into an open reading frame. In embodiments, the selected chromosome locus comprises a mutation in a gene that is correlated with a monogenic disease. In non-limiting embodiments, the indel is produced within a protein coding segment of a chromosome, at a splice junction, in a promoter, in an enhancer element, or at any other location wherein generation of an indel is desirable, provided a suitable TAM is present. In embodiments, the indel corrects a missense mutation, a frameshift mutation, or a nonsense mutation. In embodiments, the indel changes a codon for at least one amino acid in a protein coding sequence, and thus may correct a mutation in an exon. In embodiments, the indel corrects a deleterious mutation that is a component of a monogenic disorder, e.g., a disorder caused by variation in a single gene. In embodiments, an indel is 1, 2, 3, 4, or more nucleotides that are deleted or inserted.

Any component of the systems described herein can be provided on the same or different polynucleotides, such as plasmids, or a polynucleotide integrated into a chromosome. In embodiments, at least one component of the system is heterologous to the cells. In eukaryotic cells, all components of the system can be heterologous.

In embodiments, protein as described herein is introduced into the cell as a recombinant or purified protein, or as an RNA encoding the protein that is expressed once introduced into the cell, or as an expression vector, which is expressed once in the cell. In embodiments, a system of this disclosure is introduced into eukaryotic cells using, for example, one or more expression vectors, or by direct introduction of ribonucleoproteins (RNPs). In embodiments, expression vectors comprise viral vectors. In embodiments, a viral expression vector is used. Viral expression vectors may be used as naked polynucleotides, or may comprises any of viral particles, including but not limited to defective interfering particles or other replication defective viral constructs, and virus-like particles. In embodiments, the expression vector comprises a modified viral polynucleotide, including but not limited to polynucleotides from an adenovirus, a herpesvirus, or a retrovirus, such as a lentiviral vector. In embodiments, any type of a recombinant adeno-associated virus (rAAV) vector may be used. In embodiments, a recombinant adeno-associated virus (rAAV) vector may be used. rAAV vectors are commercially available, such as from TAKARA BIO® and other commercial vendors, and may be adapted for use with the described systems, given the benefit of the present disclosure. In embodiments, for producing rAAV vectors, plasmid vectors may encode all or some of the well-known rep, cap and adeno-helper components. In certain embodiments, the expression vector is a self-complementary adeno-associated virus (scAAV). Suitable ssAAV vectors are commercially available, such as from CELL BIOLABS, INC.® and can be adapted for use in the presently provided embodiments when given the benefit of this disclosure.

In embodiments, the disclosure is considered suitable for use in any eukaryotic cells, and can also be used in prokaryotic cells, such as for bioengineering prokaryotes, and for use as anti-bacterial agents. In embodiments, eukaryotic cells that are modified by the approaches of this disclosure are totipotent, pluripotent, multipotent, or oligopotent stem cells when the modification is made. In embodiments, the cells are neural stem cells. In embodiments, the cells are hematopoietic stem cells. In embodiments, the cells are leukocytes. In embodiments, the leukocytes are of a myeloid or lymphoid lineage. In embodiments, the cells are embryonic stem cells, or adult stem cells. In embodiments, the cells are epidermal stem cells or epithelial stem cells. In embodiments, the cells are cancer cells, or cancer stem cells. In embodiments, the cells are differentiated cells when the modification is made. In embodiments, the cells are mammalian cells. In embodiments, the cells are human, or are non-human animal cells. In embodiments, the non-human eukaryotic cells comprise fungal, plant or insect cells. In one approach the cells are engineered to express a detectable or selectable marker, or a combination thereof.

In embodiments, the disclosure includes obtaining cells from an individual, modifying the cells ex vivo using a system as described herein, and reintroducing the cells or their progeny into the individual for prophylaxis and/or therapy of a condition, disease or disorder. In embodiments, the cells modified ex vivo as described herein are used autologously.

In embodiments, cells modified according to this disclosure are provided as cell lines. In embodiments, the cells are engineered to produce a protein or other compound, and the cells themselves or the protein or compound they produce is used for prophylactic or therapeutic applications.

In various embodiments, the modification introduced into eukaryotic cells according to this disclosure is homozygous or heterozygous. In embodiments, the modification comprises a homozygous dominant or homozygous recessive or heterozygous dominant or heterozygous recessive mutation correlated with a phenotype or condition, and is thus useful for modeling such phenotype or condition. In embodiments a modification causes a malignant cell to revert to a non-malignant phenotype.

In certain aspects the disclosure includes a pharmaceutical formulation comprising one or more components of a system described herein. A pharmaceutical formulation comprises one or more pharmaceutically acceptable additives, many of which are known in the art. In some embodiments, the pharmaceutical compositions comprise a pharmaceutically acceptable carrier suitable for administration to humans. In some embodiments, the pharmaceutical compositions comprise a pharmaceutically acceptable carrier suitable for intraocular injection. In some embodiments, the pharmaceutical compositions comprise a pharmaceutically acceptable carrier suitable for topical application. In some embodiments, the pharmaceutical compositions comprise a pharmaceutically acceptable carrier suitable for intravenous injection. In some embodiments, the pharmaceutical compositions comprise and a pharmaceutically acceptable carrier suitable for injection into arteries. In some embodiments, the pharmaceutical composition is suitable for oral or topical administration. All of the described routes of administration are encompassed by the disclosure.

In embodiments, expression vectors, proteins, RNPs, polynucleotides, and combinations thereof, can be provided as pharmaceutical formulations. A pharmaceutical formulation can be prepared by mixing the described components with any suitable pharmaceutical additive, buffer, and the like. Examples of pharmaceutically acceptable carriers, excipients and stabilizers can be found, for example, in Remington: The Science and Practice of Pharmacy (2005) 21st Edition, Philadelphia, PA. Lippincott Williams & Wilkins, the disclosure of which is incorporated herein by reference. Further, any of a variety of therapeutic delivery agents can be used, and include but are not limited to nanoparticles, lipid nanoparticle (LNP), exosomes, and the like. In embodiments, a biodegradable material can be used. In embodiments, poly(lactide-co-galactide) (PLGA) is a representative biodegradable material. In embodiments, any biodegradable material, including but not necessarily limited to biodegrable polymers. As an alternative to PLGA, the biodegradable material can comprise poly(glycolide) (PGA), poly(L-lactide) (PLA), or poly(beta-amino esters). In embodiments, the biodegradable material may be a hydrogel, an alginate, or a collagen. In an embodiment the biodegradable material can comprise a polyester a polyamide, or polyethylene glycol (PEG). In embodiments, lipid-stabilized micro and nanoparticles can be used.

In certain approaches, compositions of this disclosure, including the described systems, and cells modified using the described systems, are used for treatment of condition or disorder in an individual in need thereof. The term “treatment” as used herein refers to alleviation of one or more symptoms or features associated with the presence of the particular condition or suspected condition being treated. Treatment does not necessarily mean complete cure or remission, nor does it preclude recurrence or relapses. Treatment can be effected over a short term, over a medium term, or can be a long-term treatment, such as, within the context of a maintenance therapy. Treatment can be continuous or intermittent.

In embodiments, a system of this disclosure is administered to an individual in a therapeutically effective amount. In embodiments, a therapeutically effective amount of a composition of this disclosure is used. The term “therapeutically effective amount” as used herein refers to an amount of an agent sufficient to achieve, in a single or multiple doses, the intended purpose of treatment. The amount desired or required will vary depending on the particular compound or composition used, its mode of administration, patient specifics and the like. Appropriate effective amounts can be determined by one of ordinary skill in the art informed by the instant disclosure using routine experimentation. For example, a therapeutically effective amount, e.g., a dose, can be estimated initially either in cell culture assays or in animal models. An animal model can also be used to determine a suitable concentration range, and route of administration. Such information can then be used to determine useful doses and routes for administration in humans, or to non-human animals. A precise dosage can be selected by in view of the patient to be treated. Dosage and administration can be adjusted to provide sufficient levels of components to achieve a desired effect, such as a modification in a threshold number of cells. Additional factors which may be taken into account include the particular gene or other genetic element involved, the type of condition, the age, weight and gender of the patient, desired duration of treatment, method of administration, time and frequency of administration, drug combination(s), reaction sensitivities, and tolerance/response to therapy. In certain embodiments, a therapeutically effective amount is an amount that reduces one or more signs or symptoms of a disease, and/or reduces the severity of the disease. A therapeutically effective amount may also inhibit or prevent the onset of a disease, or a disease relapse. In embodiments, cells modified according to this disclosure are administered to an individual in need thereof in a therapeutically effective amount.

In embodiments, the disclosure comprises providing a treatment to an individual in need thereof by introducing a therapeutically effective amount a composition of this disclosure, or modified cells as described herein to the individual, wherein the cells comprising the DNA insertion treats, alleviates, inhibits, or prevents the formation of one or more conditions, diseases, or disorders. In embodiments, the cells are first obtained from the individual, modified according to this disclosure, and transplanted back into the individual. In embodiments, allogenic cells can be used. In embodiments, the modified eukaryotic cells can be provided in a pharmaceutical formulation, and such formulations are included in the disclosure.

In embodiments, the described systems are introduced into eukaryotic cells that include but are not limited to non-human animal cells, or fungi or plant cells.

In embodiments, compositions of this disclosure are administered to avian animals, or to a canine, a feline, an equine animal, or to cattle, including but not limited to dairy cattle.

In embodiments, the disclosure includes obtaining cells from an individual, modifying the cells ex vivo using a system as described herein, and reintroducing the cells or their progeny into the individual or a immunologically matched individual for prophylaxis and/or therapy of a condition, disease or disorder, or to treat an injury, trauma or anatomical defect. In embodiments, the cells modified ex vivo as described herein are autologous cells. In embodiments, the cells are provided as cell lines. In embodiments, the cells are engineered to produce a protein or other compound, and the cells themselves and/or the protein or compound they produce is used for prophylactic or therapeutic applications.

In embodiments, eukaryotic cells made according to this disclosure can be used to create transgenic, non-human organisms.

In embodiments, one or more modified cells according to this disclosure may be used to perform a gene-drive in a population of animals, including but not necessarily limited to insects.

In embodiments, the one or more cells into which a described system is introduced comprises a plant cell. The term “plant cell” as used herein refers to protoplasts, gamete producing cells, and includes cells which regenerate into whole plants. Plant cells include but are not necessarily limited to cells obtained from or found in: seeds, suspension cultures, embryos, meristematic regions, callus tissue, leaves, roots, shoots, gametophytes, sporophytes, pollen, and microspores. Plant cells can also be understood to include modified cells, such as protoplasts, obtained from the aforementioned tissues. Plant products made according to the disclosure are included.

In embodiments, the disclosure provides an article of manufacture, which may comprise a kit. In embodiments, the article of manufacture may comprise one or more cloning vectors. The one or more cloning vectors may encode any one or combination of proteins and polynucleotides described herein. The cloning vectors may be adapted to include, for example, a multiple cloning site (MCS), into which a sequence encoding any protein or polynucleotide, such as any desired targeting RNA, may be introduced. An article of manufacture may include one or more sealed containers that contain any of the aforementioned components, and may further comprise packaging and/or printed material. The printed material may provide information on the contents of the article, and may provide instructions or other indication of how the contents of the article may be used. In an embodiment, the printed material provides an indication of a disease or disorder that is to be treated using the contents of the article.

In embodiments, when polynucleotides are delivered, they may comprise modified polynucleotides or other modifications, such as phosphate backbone modifications, and modified nucleotides, such as nucleotide analogs. Suitable modifications and methods for making nucleic acid analogs are known in the art. Some examples include but are not limited to polynucleotides which comprise modified ribonucleotides or deoxyribonucleotides. For example, modified ribonucleotides may comprise methylations and/or substitutions of the 2′ position of the ribose moiety with an ——O—— lower alkyl group containing 1-6 saturated or unsaturated carbon atoms, or with an ——O-aryl group having 2-6 carbon atoms, wherein such alkyl or aryl group may be unsubstituted or may be substituted, e.g., with halo, hydroxy, trifluoromethyl, cyano, nitro, acyl, acyloxy, alkoxy, carboxyl, carbalkoxyl, or amino groups; or with a hydroxy, an amino or a halo group. In embodiments modified nucleotides comprise methyl-cytidine and/or pseudo-uridine. The nucleotides may be linked by phosphodiester linkages or by a synthetic linkage, i.e., a linkage other than a phosphodiester linkage. Examples of inter-nucleoside linkages in the polynucleotide agents that can be used in the disclosure include, but are not limited to, phosphodiester, alkylphosphonate, phosphorothioate, phosphorodithioate, phosphate ester, alkylphosphonothioate, phosphoramidate, carbamate, carbonate, morpholino, phosphate triester, acetamidate, carboxymethyl ester, or combinations thereof. In embodiments, the DNA analog may be a peptide nucleic acid (PNA).

The following description provides examples of embodiments of the disclosure and discussion of the results. Explanations of experiments are not intended to be limiting or bound by any particular theory or interpretation.

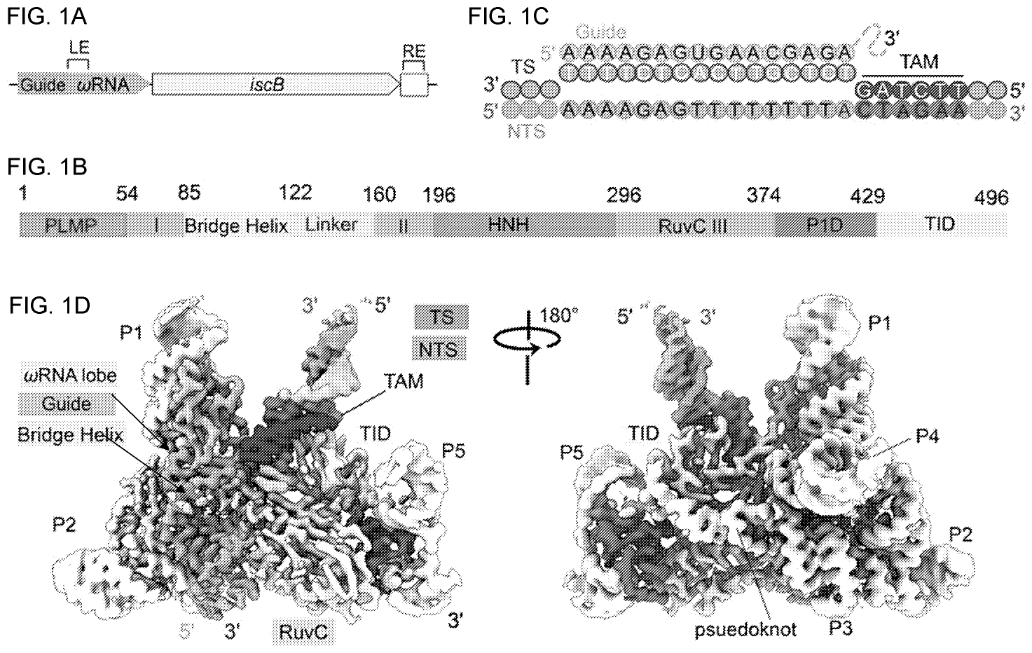

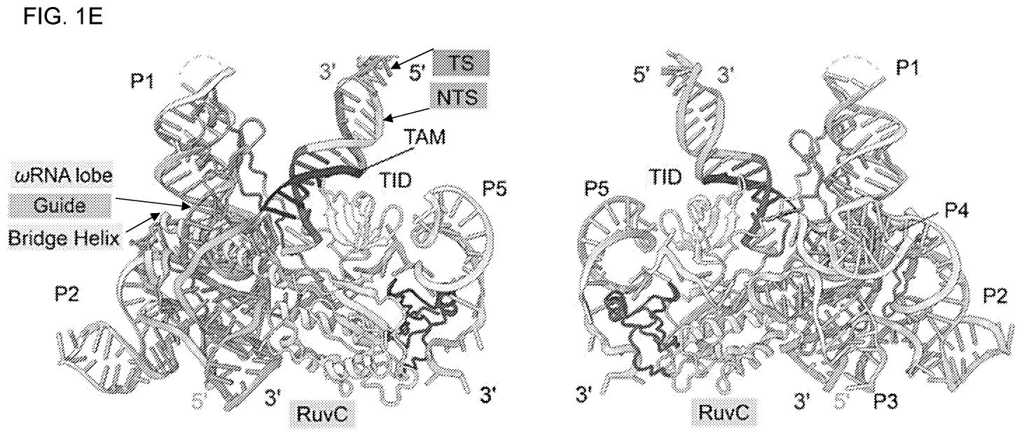

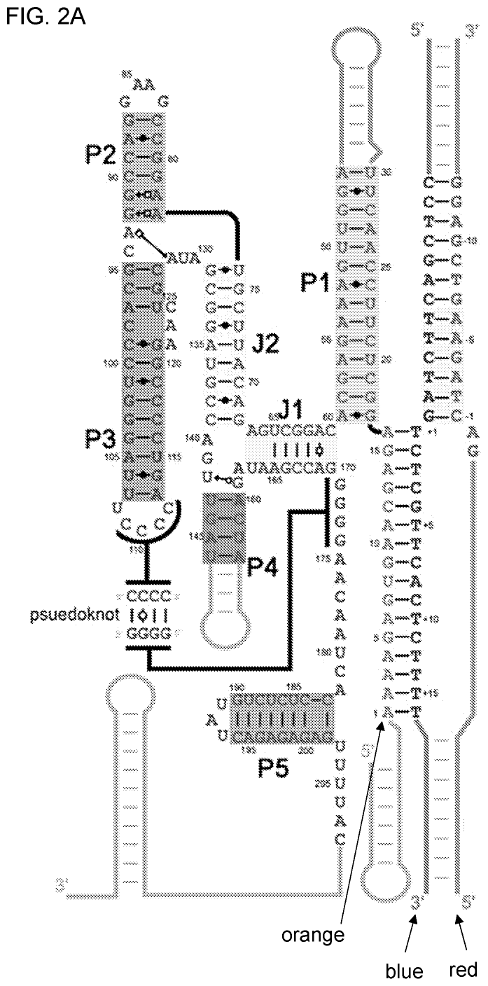

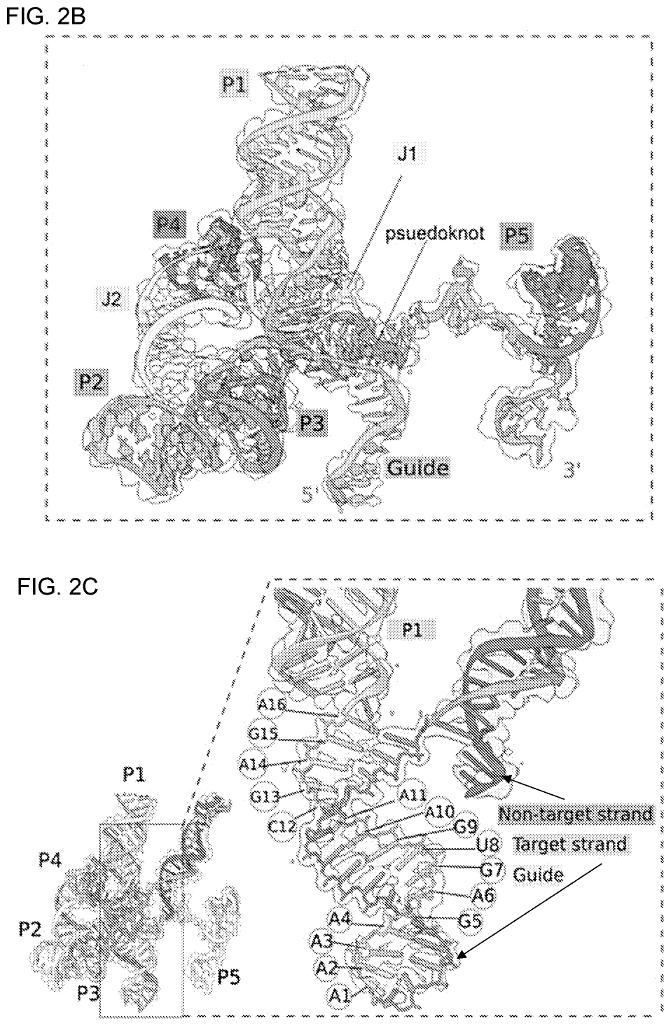

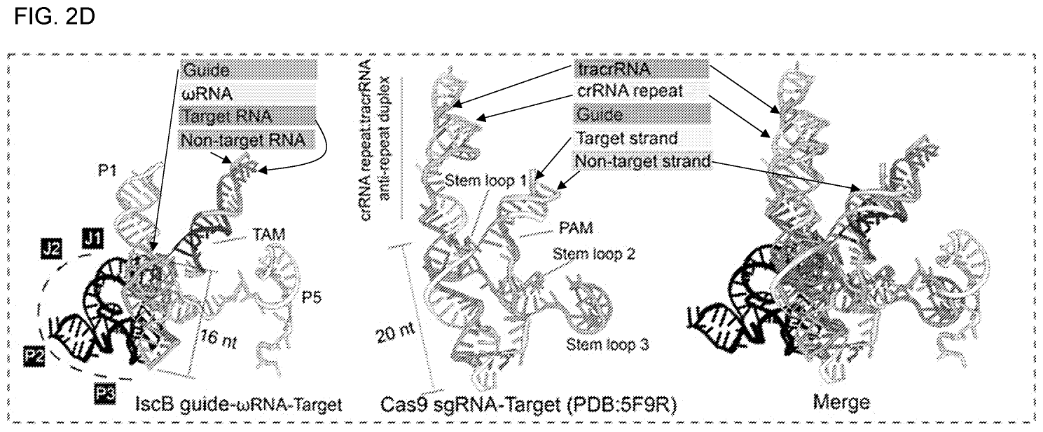

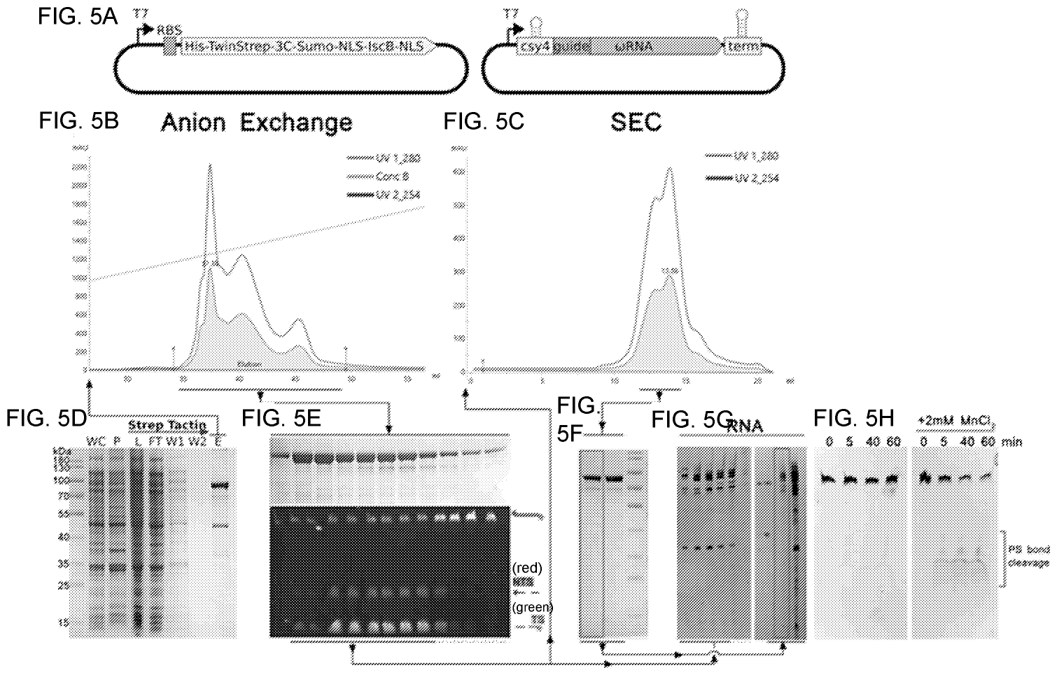

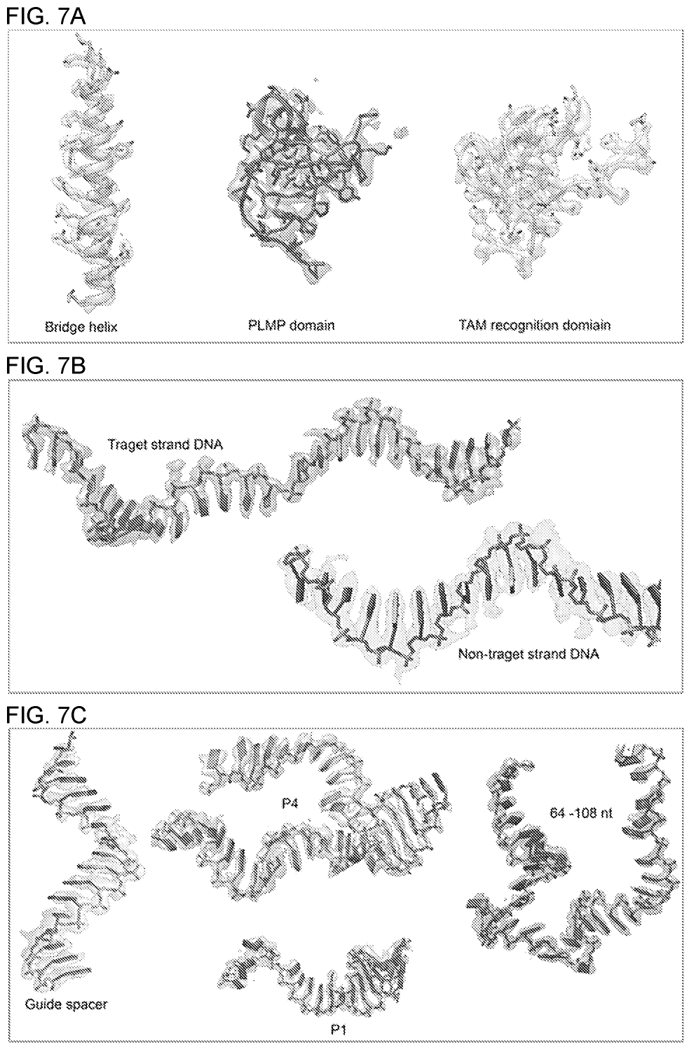

To understand the RNA-guided DNA cleavage mechanism by the compact IscB-ωRNA RNP and its relationship with Cas9-crRNA-tracrRNA, we determined a 2.78 Å structure of the gut microbiome derived OgeuIscB-ωRNA RNP complex (2) bound to target DNA using cryo-electron microscopy (cryo-EM) (FIGS. 1, FIGS. 5-7). Whereas the majority of the 496-aa IscB and 222-nt ωRNA could be unambiguously resolved, only a portion of the 60-bp DNA target could be reliably modeled. These include 13-bp of the TAM-proximal double-stranded (ds)DNA, the entire 16-nt target strand (TS) single-stranded (ss) DNA and 2-nt non-target strand (NTS) ssDNA in the R-loop region (FIG. 2A). The TAM-distal DNA is missing from the EM density due to molecular motion rather than cleavage and dissociation, because phosphorothioate modifications have been introduced into the DNA backbone at the HNH and RuvC cleavage sites (FIG. 5H) (2).

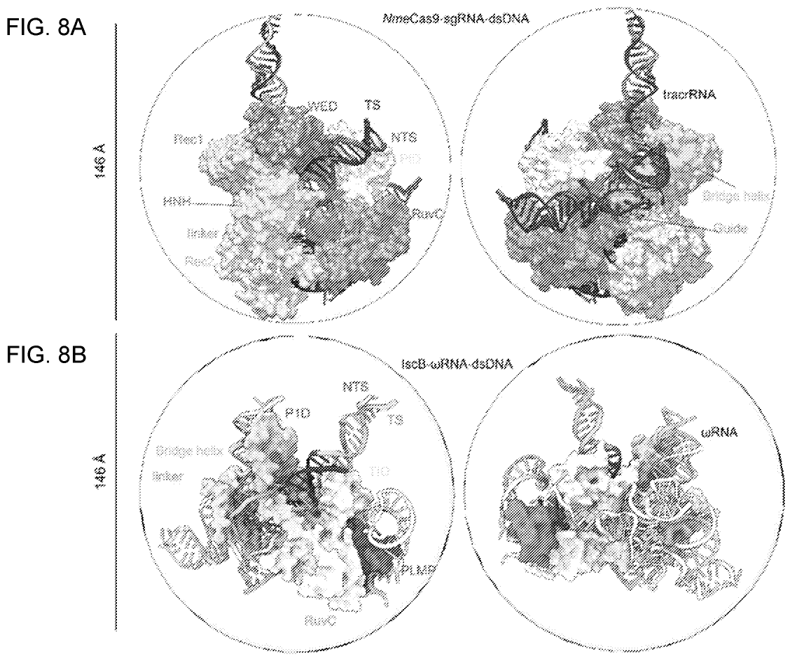

We found that the architectural organization, domain functionality, and nucleic acid binding mode are similar between IscB-ωRNA and Cas9 RNP. IscB-ωRNA adopts a similar two-lobed architecture, although its overall shape is much flatter, because several surface domains in Cas9 are missing in IscB (FIG. 8). Structural alignments revealed that the P1 stem loop of ωRNA is the functional equivalent of the crRNA repeat-tracrRNA anti-repeat duplex in the Cas9 RNP. It occupies the same location in the RNP and assists R-loop formation in a similar manner, by stabilizing the guide-RNA/TS-DNA heteroduplex through continuous base stacking (FIG. 1C-E). The TAM-containing dsDNA and the guide-RNA/TS-DNA heteroduplex in the R-loop region are accommodated by IscB-ωRNA at similar locations as in Cas9s, through conceptually similar mechanisms (FIGS. 1D-E; FIG. 8). The TS-DNA base-pairs with the 16-nt guide RNA. The first 12-bp of the DNA/RNA heteroduplex adopts a distorted A-form due to IscB contacts, with a widened major groove and base-stacking almost perpendicular to the helical axis. The last 4-bp of the heteroduplex adopts a canonical A-form geometry (FIG. 1D-E).

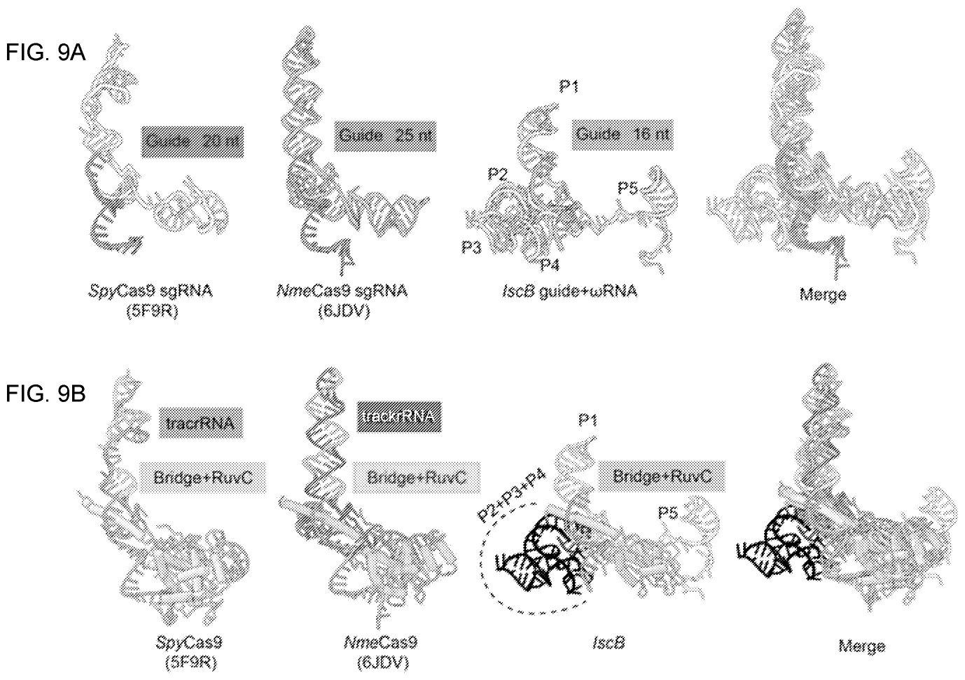

Architecturally, a main structural difference between IscB and Cas9 is its lack of a polypeptide-based recognition (REC) lobe (FIG. 8). The functional replacement is the ωRNA lobe (from J1 to the pseudoknot), which folds into a sophisticated tertiary RNA structure (FIG. 2A). The structured portion of ωRNA was previously identified as HEARO RNA (HNH Endonuclease-Associated RNA and ORF) (17). This RNA and its associated HNH-containing ORF together was speculated to constitute a mobile genetic element (17). The presently provided 3D structure is consistent with previous the secondary structure models (2, 17). The central portion of ωRNA is a tail-to-tail stacked P2-P3 superhelix. J2 helix extrudes from the P2-P3 junction, then bifurcates into P4 and J1 at its end. While P4 projects away, J1 projects towards the apex of P3. The following residues zip up with the apical loop of P3 through a 4-bp G/C-rich pseudoknot (FIG. 2A-B). Following the pseudoknot, ωRNA extends horizontally along the backside of the IscB as a conserved ss-linker and a terminator-like element (P5, followed by four consecutive Us) (FIG. 2B). A conserved and highly structured RNA typically mediates either catalysis, ligand binding, or RNP formation (17). The presently described structure does not support a direct involvement of ωRNA in RNA-guided DNA cleavage because the bulk of ωRNA is insulated from the guide-RNA/TS-DNA heteroduplex by a layer of protein elements from IscB (FIGS. 1C-D). The presently described structure further suggests the evolutionary trend from ancestral IscB to Cas9 involves replacing the structural roles of ωRNA with protein domains. However, the crRNA-tracrRNA of SpCas9 and NmeCas9 RNPs still contain structural elements reminiscent of P1, J1, pseudoknot, and terminator in ωRNA (FIG. 2D, FIG. 9) presumably because these elements are indispensable for RNP assembly.

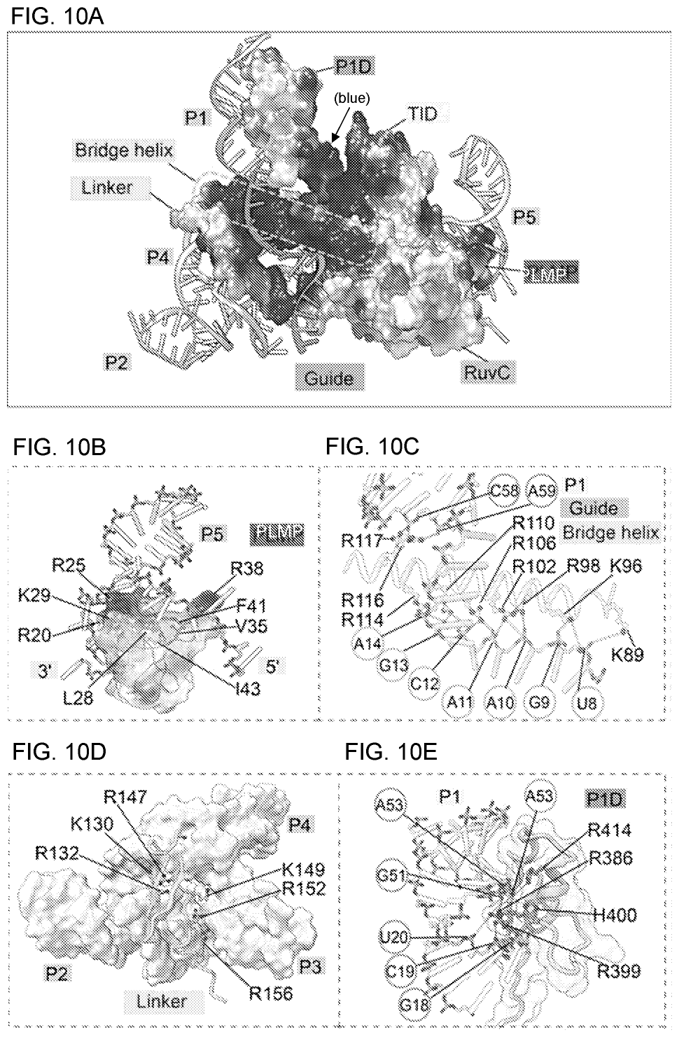

Opposite from the ωRNA lobe, the equivalent of the Cas9 nuclease (NUC) lobe contains the RuvC nuclease as its platform. RuvC is woven together from three split polypeptide elements (FIGS. 1B, FIG. 10A). It projects structural domains to various regions of the RNP. These elements are rich in positive surface charges, making favorable contacts with nucleic acids in different regions (FIG. 10A). The N-terminal PLMP motif-containing domain is packed at the edge of the NUC lobe to capture the terminator-like structure in ωRNA (FIG. 10B). The Arg-rich bridge helix is regarded as one of the most conserved structural elements in Cas9 (7, 8). It plays an equally important function in IscB-ωRNA RNP. Projected from RuvC, the bridge helix travels underneath the guide RNA, along the pseudoknot and J1, and at the base of P1, making multiple electrostatic contacts to the sugar-phosphate backbones. A line of consecutive arginine and lysine residues along one phase of the bridge helix make consecutive phosphate contacts to seven residues in the RNA guide (U8-A14), immobilizing the seed region of the guide in place for TS-DNA base-pairing (FIG. 10C). A β-hairpin followed by a flexible linker connects the bridge helix back to RuvC. Although very degenerate in size and structural complexity, this flexible structural elements “glues” ωRNA and middle portion of the guide RNA together with its positive Arg/Lys residues (FIG. 10D). The HNH nuclease domain is projected internally from RuvC. Like in many Cas9s, this domain is not well resolved in the averaged EM density map due to conformational flexibility. RuvC sends P1D domain to recognize the P1 helix of ωRNA; its functional equivalence is the WED domain in Cas9 (FIGS. 10E, FIG. 8) (7, 10, 16). Finally, P1D connects with the TAM-interaction domain (TID) situated above RuvC through flexible linkers.

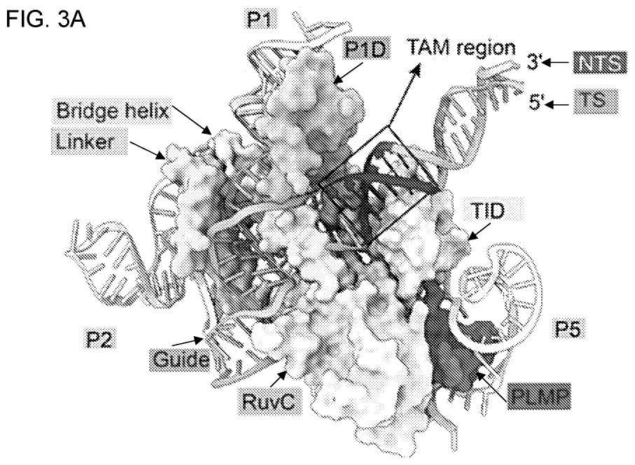

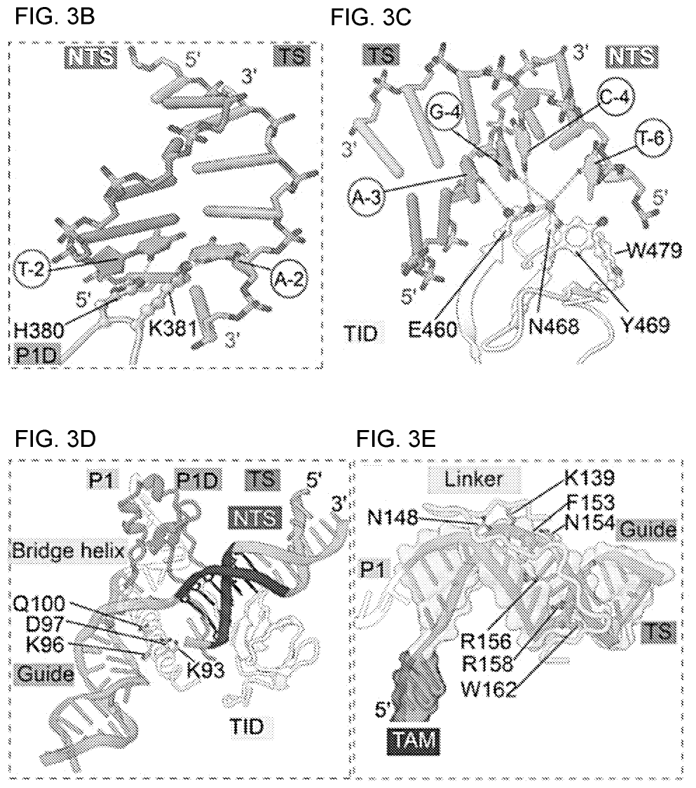

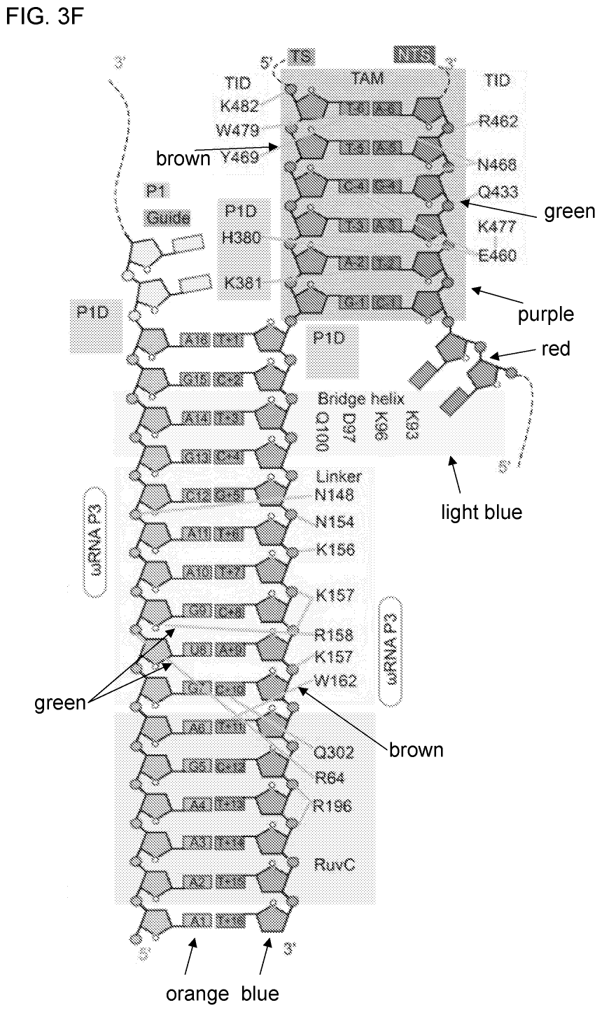

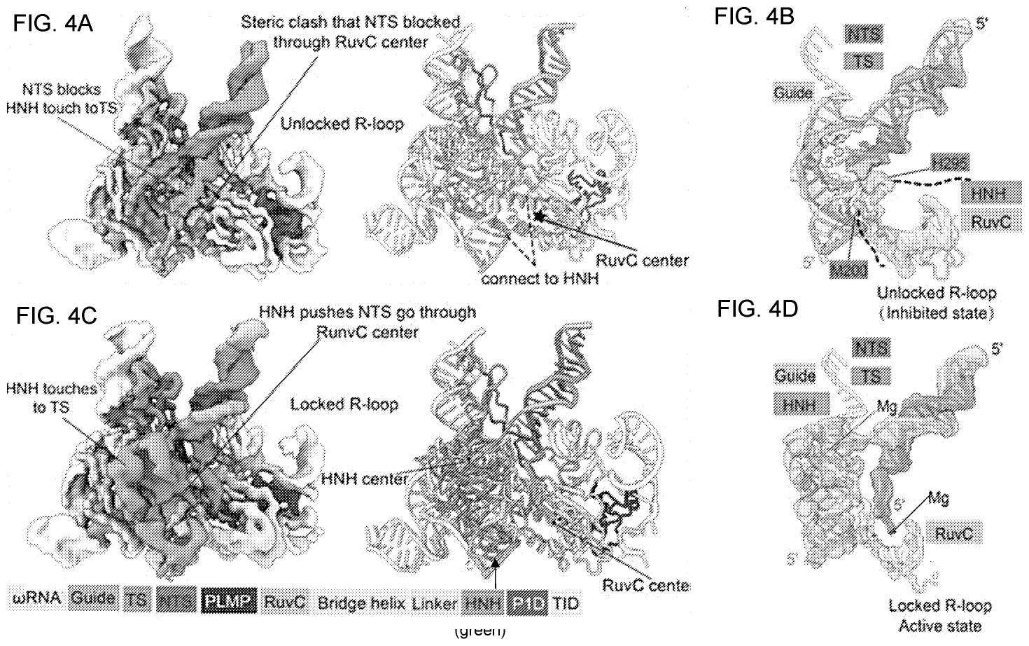

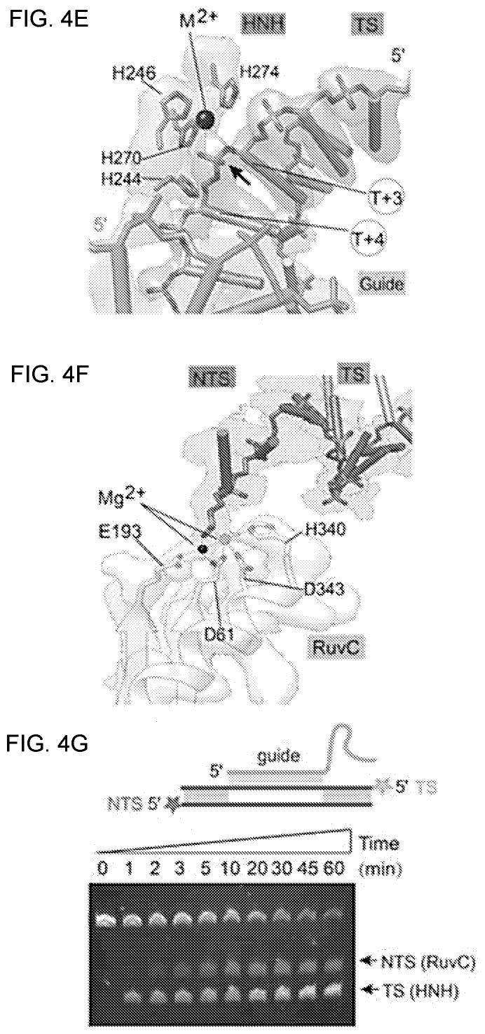

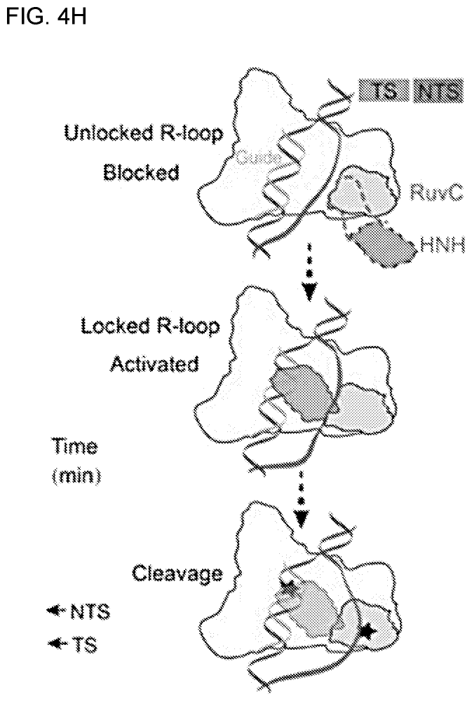

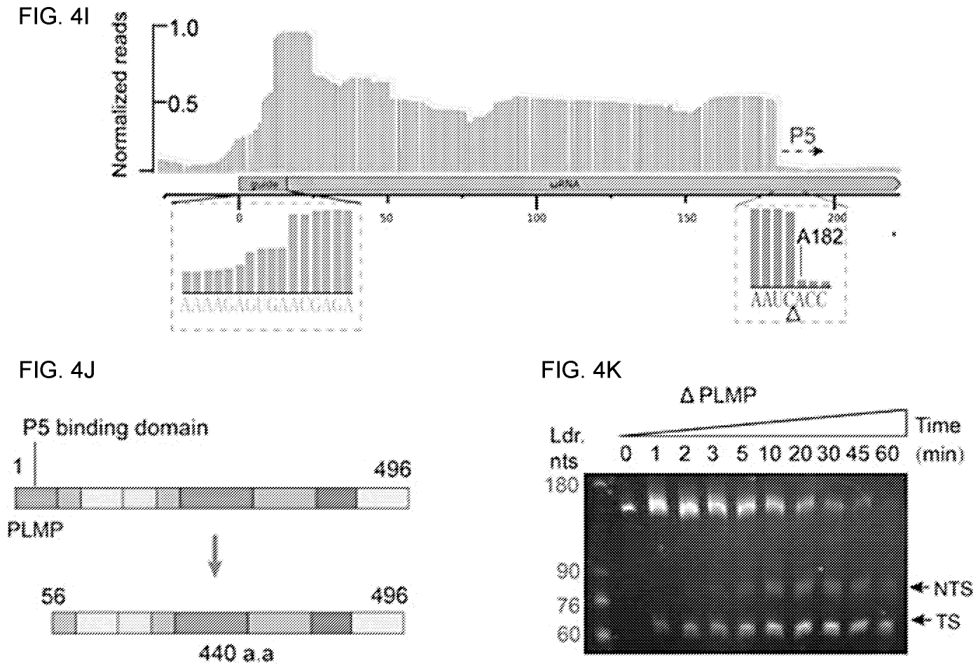

The OgeuIscB-ωRNA/R-loop structure explains the RNA-guided target recognition mechanism in high resolution (FIG. 3A). TAM (5′-NWRRNA-3′ (2); actual sequence: CTAGAA) in the dsDNA target is captured from the major groove side by the TID domain of IscB and from the minor groove side by the P1D linker (FIGS. 3B, 4C). No contact was found at −1 TAM position. The −2 TAM position is recognized from the minor groove side by His397 and K380 in P1D linker to O2 of TNTS-2 and N3 of ATS-2, respectively. G-C pairs may be rejected in either combination due to the steric clash caused by the N2 protrusion from guanosine into the minor groove. The −3 and −4 of TAM appear to be probed indirectly for shape complementarity. It is believed that only purines in the NTS support the Van der Waals contacts to the backbone of Glu459 and Gly460 in TID; pyrimidines are too recessed. The −6 TAM position is recognized through hydrophobic contacts to the methyl groups of TTS-6 in the major grove, by Tyr468 and Trp478 in TID, respectively. Many IscB homologs encode smaller TID domains and specify less stringent TAM codes (2). Domain swapping attempts, structure-guided design, and directed evolution as described herein provide more versatile IscB-ωRNA tools and may provide for use of expanded TAM codes.

A recent Cas9 study showed that off-targeting is inversely correlated with the extent of protein contacts to the guide-RNA/TS-DNA heteroduplex; the more local interactions to specify an A-form geometry, the less mismatch tolerance therein (14). In this regard, the present structural analysis identified extensive R-loop contacts (FIG. 3A,,4D4D-F), which implies that IscB-ωRNA can specify a DNA target stringently despite its miniature size and shorter R-loop specification. A P1D loop (aa 396-408) specifies the first two base-pairs of the guide-RNA/TS-DNA heteroduplex from the minor groove side. The bridge helix and the following β-hairpin and linker specifies the middle portion of the heteroduplex (bp 2-9) from major and minor sides, respectively. ωRNA provides the platform support for these contacts, and a portion of the ωRNA backbone (P2, nt 114-116) directly contacts the backbone of guide RNA (bp 10-11). The RuvC domain then contacts the minor groove of bp 9-13. Basepairs 14-16 are not contacted and have weaker density. As described below, this region is recognized when HNH docks onto the DNA/RNA heteroduplex.

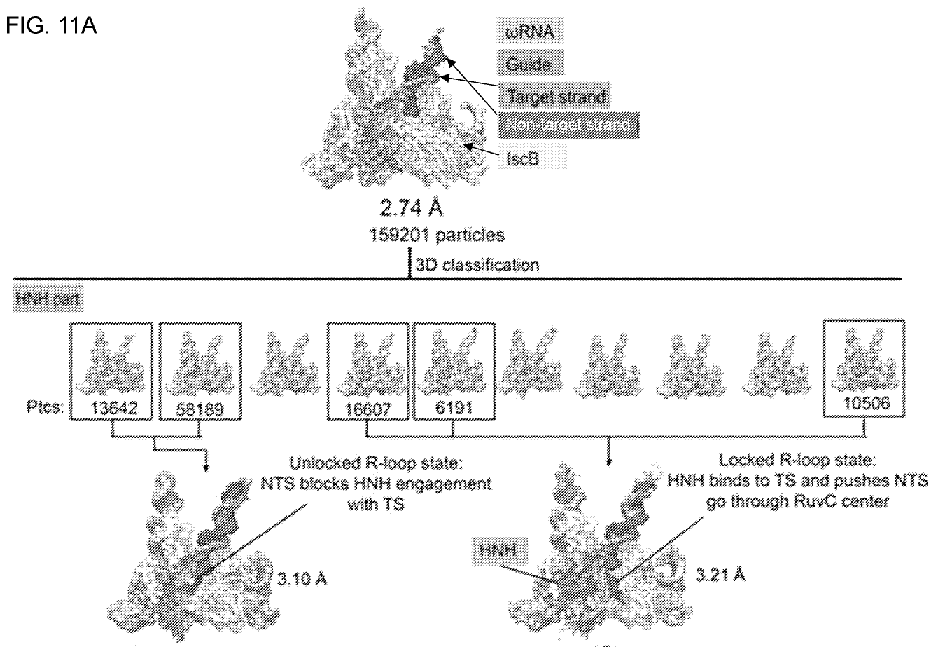

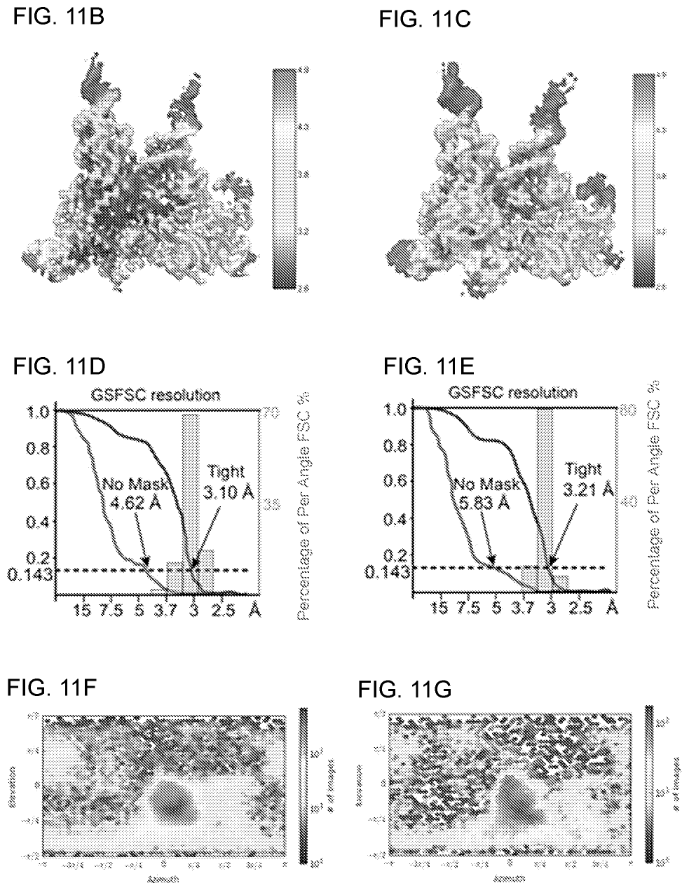

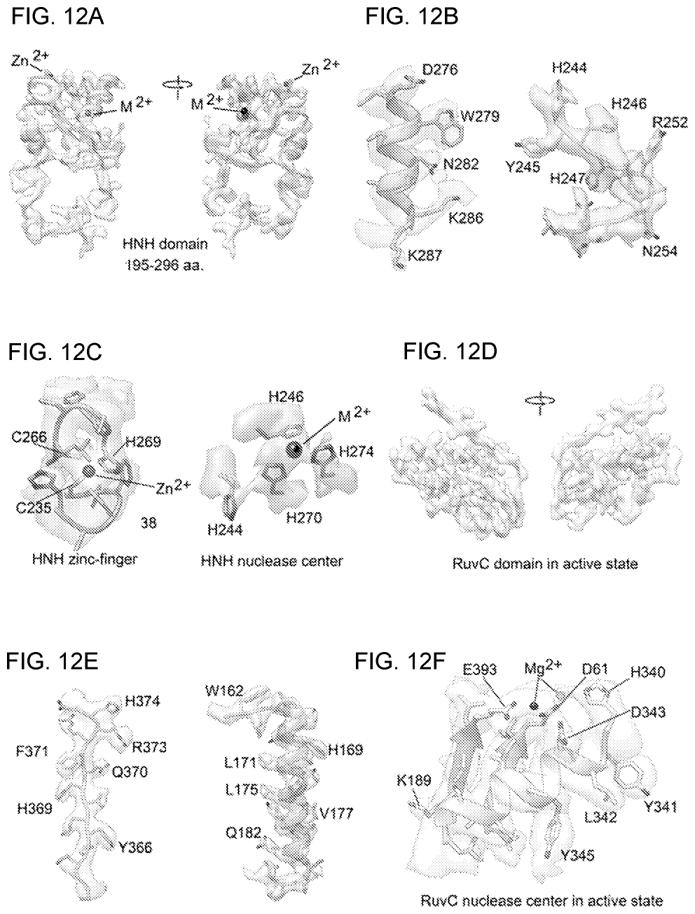

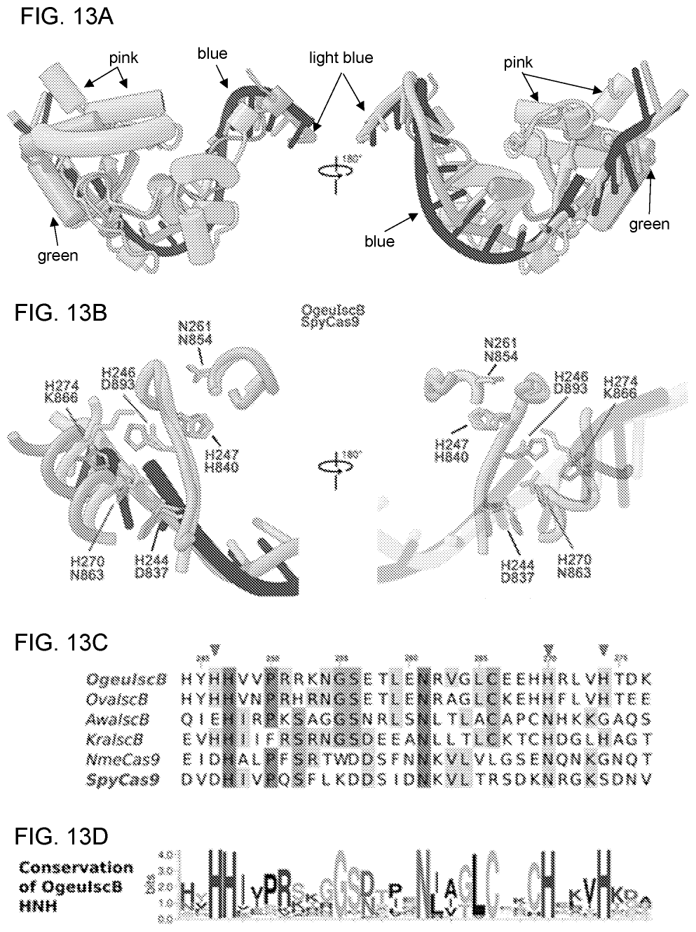

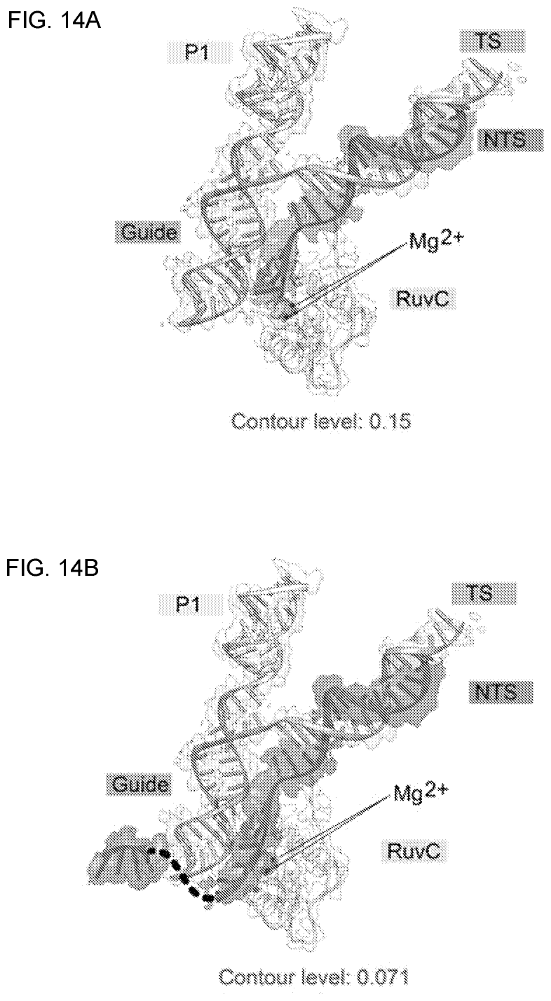

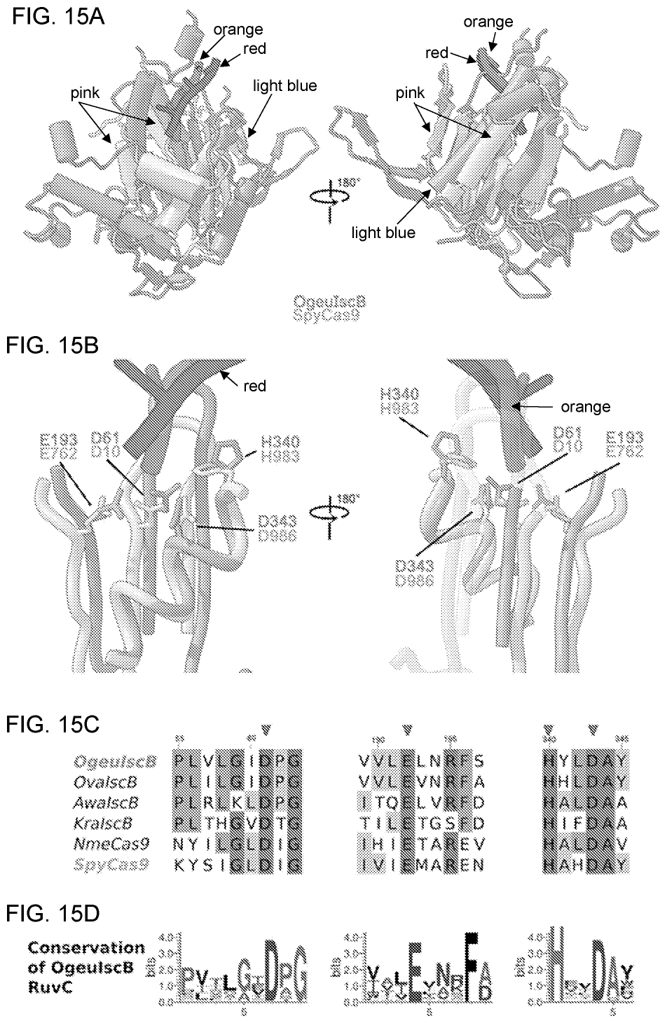

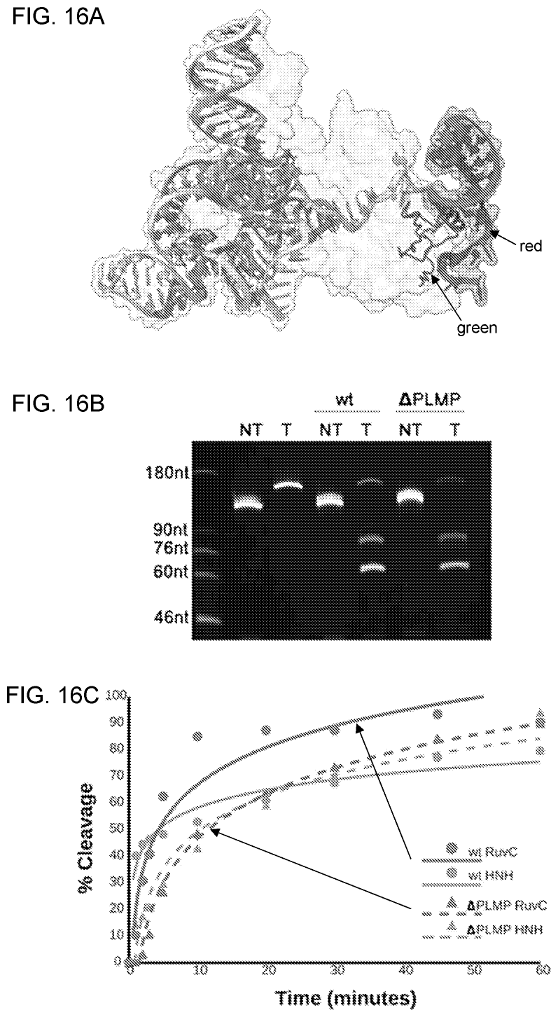

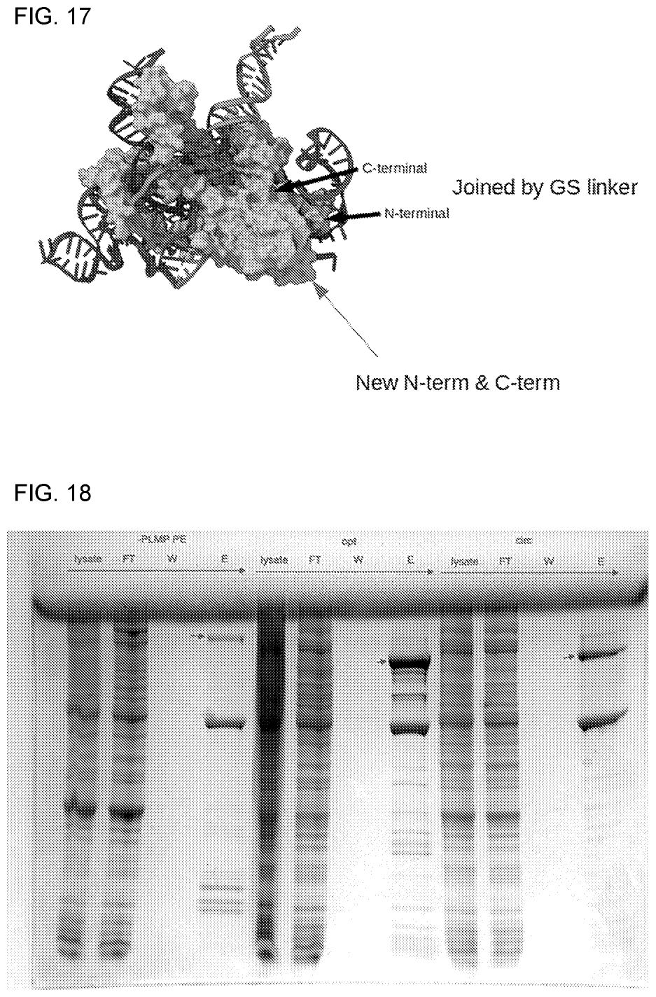

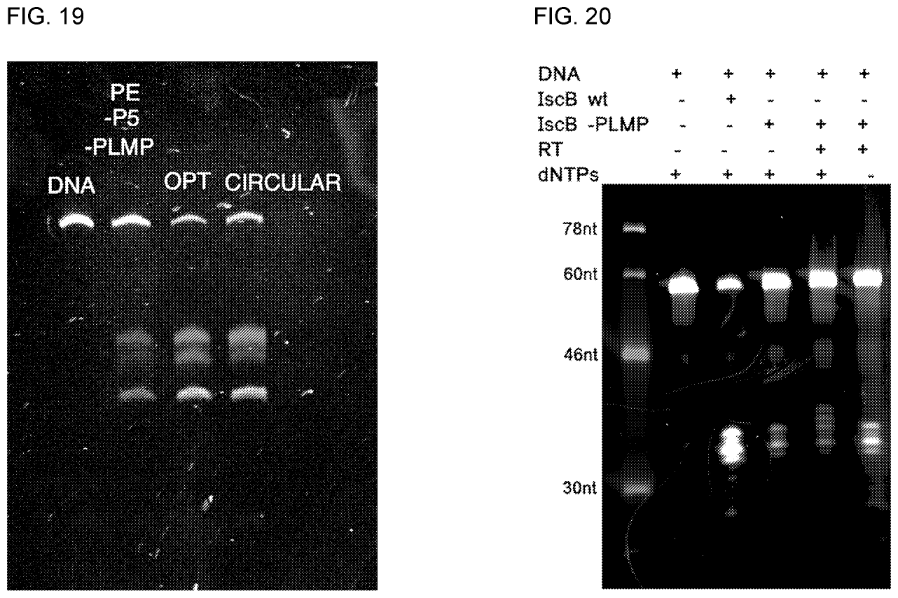

To analyze the DNA cleavage mechanism, we analyzed the conformational dynamics in the IscB-ωRNA/R-loop EM reconstruction. Finer conformational sampling revealed two predominant conformational states. In the unlocked R-loop state (FIGS. 4A-B, FIG. 11), the 3.1 Å map shows the NTS-DNA traveling near the RNA-bound TS-DNA. NTS-DNA is blocked from accessing the RuvC active site due to a steric clash with the anchor connecting HNH to RuvC (FIG. 4A). Although unresolved in EM density, HNH is likely part of the blocking mechanism as well. Its approximate location can be inferred by comparing it to the NmeCas9 apo structure (12). In contrast, the 3.2 Å locked R-loop state (FIG. 4C-D) shows HNH docking onto the RNA/TS-DNA heteroduplex and caging it with the rest of the IscB elements mentioned previously (FIG. 3A). The entry and exiting linkers from RuvC to HNH probe for shape complementarity with the bottom and middle portions of the DNA/RNA heteroduplex, respectively. The body of HNH sinks into the major groove of the DNA/RNA heteroduplex (FIG. 4C). These close contacts are expected to further reduce mismatch tolerance. An Alphafold (18) predicted HNH structure was docked into EM map (FIG. 4C, 4D, FIG. 12). While the HINH core structure agreed with the density very well, manual adjustments were needed to fit the predicted linker structures into density (FIG. 12). The HNH nuclease “bites” onto the sugar-phosphate backbone of TS-DNA in the heteroduplex. The His-rich active site coordinates a catalytic metal ion towards the phosphate of the 4th residue in TS-DNA (FIG. 4E, FIG. 13), which would leave 3-nt at the TS-DNA side after cleavage, consistent with the biochemistry (2). Topologically, and without intending to be constrained by any particular interpretation, it is considered that the observed docking movement is only possible if HNH passes underneath NTS-DNA, which in turn clears the roadblock that previously denied NTS access to RuvC. A continuous corridor of density reveals TAM-proximal NTS-DNA entering the RuvC active site, coordinated by a metal ion therein (FIG. 4F). The order of events explains the biochemical observation that TS-DNA cleavage precedes the NTS cleavage (FIG. 4G-H). Previously, RuvC in SpCas9 was found to be allosterically controlled by HNH conformational changes (19), and its cleavage rate trails behind HNH (20). The present structural analysis defines the structural basis for the allosteric control in IscB (FIG. 4H). The same mechanism is likely present in Cas9 RNP.

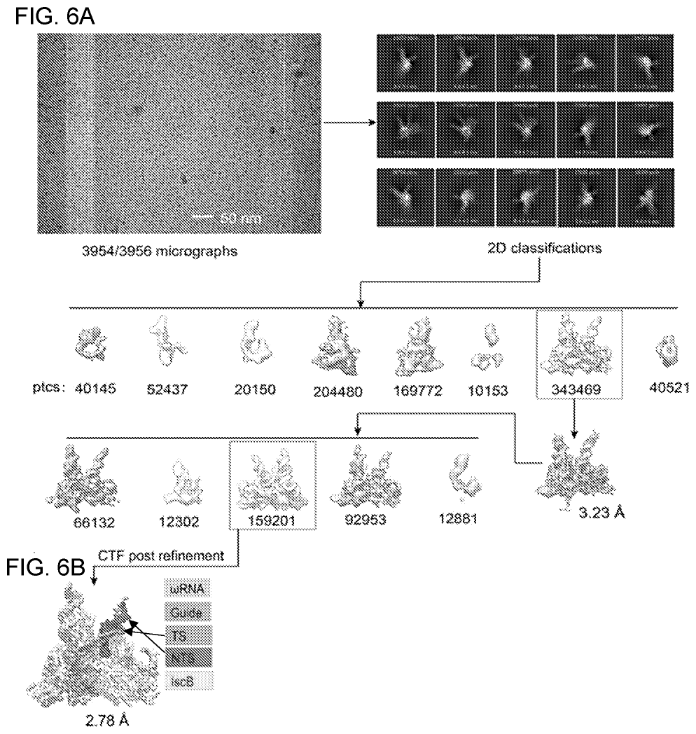

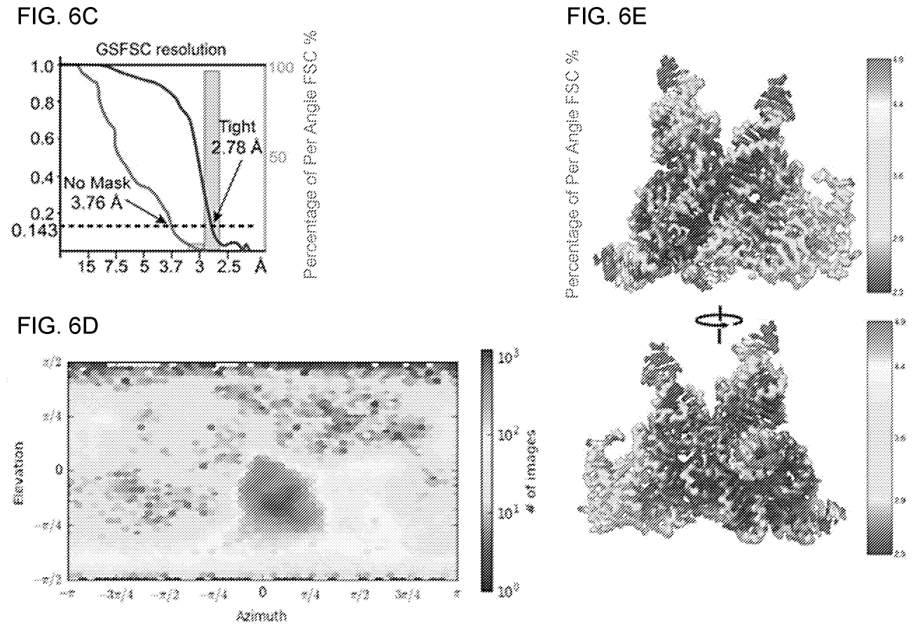

Given the robust RNA-guided DNase activity in vitro, it is puzzling to observe only weak genome editing activity from OgeuIscB-ωRNA in human cells in previous reports (2). We noticed the presence of multiple RNA species in the purified OgeuIscB-ωRNA RNP and subjected the sample for RNA deep-sequencing. The sequence coverage dropped immediately before the terminator-like P5 element of ωRNA (FIG. 4I). This is surprising because P5 density is clearly present in the RNP structure. We analyzed whether the cryo-EM particle picking and 3D reconstruction process might have inadvertently biased towards P5-containing single particles (FIG. 6A). Given the high DNA cleavage activity in a presently described OgeuIscB-ωRNA RNP, we analyzed whether the PLMP-P5 interaction may be dispensable for RNA-guided DNA cleavage. Indeed, OgeuIscB-ωRNA with a structure-guided PLMP domain truncation (Δaa1-55) was only slightly slower than the wild-type RNP in target DNA cleavage (FIG. 4J-K, FIG. 16). This result indicates that the PLMP domain is not ubiquitously essential for RNA-guided DNA cleavage among IscB homologs (2), and may be removed or repositioned as described above, or be activated by gain of function mutations as described herein. It is considered, without intending to be constrained by any particular interpretation, that the PLMP-P5 interaction may instead be important for the biogenesis of IscB-ωRNA, by controlling the readthrough and termination ratio at ωRNA P5 in order to achieve copy number balance between IscB and ωRNA. Alternatively, these domains may be important for the transposition of IS200/IS605. The sequencing result further revealed a stepwise decrease in coverage for the guide (after the 6th and 10th nucleotide; FIG. 4I). This pattern is consistent with the observed guide accessibility in the IscB-ωRNA structure (FIG. 1). Naturally occurring tracrRNA variants containing a 11-nt-long guide were shown to convert SpCas9 from a nuclease to an RNA-guided transcriptional repressor (21). Chemical modification efforts also revealed that the guide RNA integrity could influence the in vivo activity of Cas9 significantly (22).

In view of the foregoing, it will be recognized that the present structural analysis provides a high-resolution explanation for the relationship between IscB-ωRNA and Cas9-crRNA-tracrRNA. The disclosure supports the described genome editing tools, packageable into AAV. As demonstrated above, fifty-five amino acids have already been removed from IscB without abolishing its activity (FIG. 41), thereby supporting the presently described approached when delivered using recombinant viral vectors, such as AAV.

### Materials and Methods

An IscB from a human gut metagenome (Genbank: OGEU01000025.1, CDS: 120729-122219) was codon optimized and synthesized (GeneUniversal) with an N-terminal 6× His, thrombin, Twin-Strep-tag, HRV 3C protease site, sumo protease site, SV40NLS and C-terminal nucleoplasm NLS. This IscB construct was cloned into pCDFDuet™-1 (Novagen) vector between the Ncol and BamHI sites. The IscBAPLMP expression vector was constructed using PCR mutagenesis using F_remove_PLMP CTGGTTCTGGGTATTGATCCG (SEQ ID NO:19) and R_remove_PLMP AGATCCCACCTTCCGTTTC (SEQ ID NO:20). The ωRNA (Genbank: OGEU01000025.1, 120523-120728) sequence was synthesized (GeneUniversal) and cloned into pUC57-Kan between the HindIII and EcoRI sites. Upstream of the ωRNA was a T7 promoter, csy4 stem loop, and 16nt guide. A T7 terminator was placed downstream of the ωRNA.

IscB and ωRNA plasmids were co-transformed into E. coli T7 Express cells (New England Biolabs). The cell culture was grown in LB medium supplemented with 0.75 g L-cysteine/L at 37° C. until the optical density at 600 nm reached 0.8. Expression was induced by adding isopropyl-β-D-thiogalactopyranoside (IPTG) to a final concentration of 0.5 mM at 16° C. overnight. Cells were collected by centrifugation and lysed by sonication in buffer A (175 mM NaCl, 50 mM HEPES pH7.25, 2 mM TCEP, 5% glycerol, 2.5 mM MgC12) with 1 mM phenylmethylsulfonyl fluoride (PMSF). The lysate was centrifuged at 12,000 r.p.m. for 60 minutes at 4° C., and the supernatant was applied onto a pre-equilibrated strep-tactin resin (iba lifesciences). Resin was then washed with 15 mL of buffer A, 25 mL of buffer A with 0.1 mM CaCl2 and 2 μg DNaseI (Gold Biotechnology), 20 mL buffer B (1M NaCl, 50 mM HEPES pH7.25, 2 mM TCEP, 2.5 mM MgC12), and 40 mL buffer A. Resin was resuspended in buffer A and incubated with 3C protease at 4° C. overnight. The flow through buffer containing the 3C cleaved IscB was then concentrated and further purified by anion chromatography (MonoQ 5/50GL; Cytiva) with a gradient elution beginning with buffer A and increasing the percent of buffer B. Peak fractions were tested for cleavage activity and pooled. Pooled fractions were concentrated and further purified by size-exclusion chromatography (Superdex 200 Increase 10/300 GL; Cytiva) equilibrated with buffer C (175 mM NaCl, 50 mM HEPES pH7.25, 2 mM TCEP, 2.5 mM MgC12). The first peak was collected, concentrated, and flash frozen with liquid nitrogen.

### DNA Substrate Preparation

DNA oligonucleotides for cryo-EM were synthesized (Integrated DNA Technologies). NT_Fam_03_target_PS/56-FAM/CGCCCCACGAGGGTACGGCAAAAGA*G*T*T*T*T*T*TTTACTAGAAGTCGA GGTCAGCCCGTGGC (SEQ ID NO:21), T_03_target_PS GCCACGGGCTGACCTCGACTTCTAGT*C*T*C*G*T*T*CACTCTTTTGCCGTACCCT CGTGGGGCG (SEQ ID NO:22) (*phosphorothioate bond). Oligonucleotides were annealed in duplex buffer (30 mM HEPES pH 7.5, 100 mM potassium acetate) by heating to 95° C. for 5 min and slowly cooling. Annealed Oligonucleotides were purified in a 10% native PAGE gel. The template strand for DNA cleavage assays was synthesized by (Integrated DNA Technologies) Template_cleavage_target CCCACGAAGGGTTACGGCAAAGCATCATCAAAAAGAGTGAACGAGACTAGAAGT CTGAAAAGGTCATTTTTTAAAGCC (SEQ ID NO:23). DNA substrate for cleavage assays was produced using PCR using F_cleavage_target /Cy3/CCGCAAGAGGATGATTCGGGTGCGGCAACGGAAGGGGAGGGCCCCACGAA GGGTTACGG (SEQ ID NO:24) R_cleavage_target /Cy5/GCTGATCTGATGCAGTTAAGTGCCTGCTGGGCTTTAAAAAATGACCTTTTCA GAC (SEQ ID NO:25). PCR products were agarose gel purified using GeneJet gel extraction kit (Thermo Scientific).

### Cleavage Assays

The cleavage assays were performed as follows. 10 μL reactions were prepared where 20 nM target DNA was incubated with 1 μM IscB in cleavage buffer (50 mM NaCl, 50 mM HEPES pH7.25, 2 mM BME, 5 mM MgCl2) and incubated at 37° C. for 1 hour. For time course experiment reactions were quenched with the addition of EDTA to 150 mM (final concentration) and an equal volume of 100% formamide. 2 mM MnCl2 was added to the cleavage buffer for phosphorothioate bond cleavage rescue experiment. Samples were heated to 95° C. for 10 minutes and run on 12% urea-PAGE. Fluorescent signals were imaged using ChemiDoc (BioRad) and quantified using Image Lab.

### RNA Extraction, Urea Gel Running, and RNA Sequencing

20 μL of IscB sample and 20 μL phenol-chloroform solution was mixed together and vortexed vigorously at room temperature. The aqueous and organic phases were separated by 13,000 rpm centrifuge for 2 minutes at room temperature. 10 μL sample was taken from the aqueous phase (top layer), mixed with 10 μL of formamide loading dye, heat-denatured at 95° C. for 10 min, and immediately loaded to a 12% urea-polyacrylamide (PAGE) gel. After 50 minutes of electrophoresis at 25 watts, the gel was stained with EtBr to for 10 min, destained in water for 10 minutes, and scanned with the ChemiDoc imaging system (Bio-Rad) at appropriate wavelength.

### Small RNA Sequencing

Phenol-choloroform extracted RNA was ethanol precipitated with 9× volumes of chilled 100% ethanol and 1 μL of GlycoBlue (Invitrogen) and stored at −80° C. Precipitated RNA was centrifuged at 13,000 rpm for 30 minutes at 4° C. Ethanol was removed, the RNA pellet was dried, and resuspended in nuclease free water. RNA was sent for the Cornell TREx facility for NEBNext small RNA library prep and Illumina sequencing. Library was sequenced to a depth of 10 million reads with a read length of 75 nt. The Cornell TREx facilities processed the raw single-end reads with trim-galore package to trim low quality bases and adapter sequences. Trimmed reads were aligned to the T7 Express E. coli genome (Genbank: CP014268.2) and IscB expression plasmids using STAR v2.7. BAM files were visualized in Integrated Genome Browser (IGV).

### Cryo-EM Sample Preparation, Data Acquisition, and Processing

IscB was incubated for 15 minutes at 37° C. with the target DNA in cleavage buffer. DNA was supplied at a 3 fold molar excess to IscB (0.5 mg/mL final concentration). 3.5 μL of were applied to a Quantifoil holey carbon grid (1.2/1.3, 200 mesh) which had been glow-discharged with 20 mA at 0.39 mBar for 30 seconds (PELCO easiGlow). Grids were blotted with Vitrobot blotting paper (Electron Microscopy Sciences) for 6.5 s at 4° C., 100% humidity, and plunge-frozen in liquid ethane using a Mark IV FEI/Thermo Fisher Vitrobot. Data were collected on a Krios G3i Cryo Transmission Electron Microscope (Thermo Scientific) with a Ceta 16M CMOS camera 300 kV, Gatan K3 direct electron detector. The total exposure time of each movie stack led to a total accumulated dose of 50 electrons per Å2 which fractionated into 50 frames. Dose-fractionated super-resolution movie stacks collected from the Gatan K3 direct electron detector were binned to a pixel size of 1.1 Å. The defocus value was set between −1.0 μm to −2.5 μm.

Motion correction, CTF-estimation, blob particle picking, 2D classification, 3D classification and non-uniform 3D refinement were performed in cryoSPARC v.2 (28). Refinements followed the standard procedure, a series of 2D and 3D classifications with C1 symmetry were performed as shown in FIG. 6 to generate the final maps. A solvent mask was generated and was used for all subsequent local refinement steps. CTF post refinement was conducted to refine the beam-induced motion of the particle set, resulting in the final maps. The detailed data processing and refinement statistics for cryo-EM structures are summarized in FIG. 6 and FIG. 11.

The following reference listing is not an indication that any reference is material to patentability.
1. Kapitonov V V, Makarova K S, Koonin E V, ISC, a Novel Group of Bacterial and Archaeal DNA Transposons That Encode Cas9 Homologs. J Bacteriol 198, 797-807 (2015).
2. Altae-Tran H et al., The widespread IS200/IS605 transposon family encodes diverse programmable RNA-guided endonucleases. Science 374, 57-65 (2021).
3. Karvelis T et al., Transposon-associated TnpB is a programmable RNA-guided DNA endonuclease. Nature 599, 692-696 (2021).
4. Jinek M et al., A programmable dual-RNA-guided DNA endonuclease in adaptive bacterial immunity. Science 337, 816-821 (2012).
5. Cong L et al., Multiplex genome engineering using CRISPR/Cas systems. Science 339, 819-823 (2013).
6. Gasiunas G, Barrangou R, Horvath P, Siksnys V, Cas9-crRNA ribonucleoprotein complex mediates specific DNA cleavage for adaptive immunity in bacteria. Proc Natl Acad Sci U S A 109, E2579-2586 (2012).
7. Nishimasu H et al., Crystal structure of Cas9 in complex with guide RNA and target DNA. Cell 156, 935-949 (2014).
8. Shmakov S et al., Discovery and Functional Characterization of Diverse Class 2 CRISPR-Cas Systems. Mol Cell 60, 385-397 (2015).
9. Jiang F et al., Structures of a CRISPR-Cas9 R-loop complex primed for DNA cleavage. Science 351, 867-871 (2016).
10. Jinek M et al., Structures of Cas9 endonucleases reveal RNA-mediated conformational activation. Science 343, 1247997 (2014).
11. Nishimasu H et al., Crystal Structure of Staphylococcus aureus Cas9. Cell 162, 1113-1126 (2015).
12. Sun W et al., Structures of Neisseria meningitidis Cas9 Complexes in Catalytically Poised and Anti-CRISPR-Inhibited States. Mol Cell 76, 938-952 e935 (2019).
13. Das A et al., The molecular basis for recognition of 5′-NNNCC-3′ PAM and its methylation state by Acidothermus cellulolyticus Cas9. Nat Commun 11, 6346 (2020).
14. Bravo JPK et al., Structural basis for mismatch surveillance by CRISPR-Cas9. Nature 603, 343-347 (2022).
15. Mojica F J M, Diez-Villasenor C, Garcia-Martinez J, Almendros C, Short motif sequences determine the targets of the prokaryotic CRISPR defence system. Microbiology (Reading) 155, 733-740 (2009).
16. Anders C, Niewoehner O, Duerst A, Jinek M, Structural basis of PAM-dependent target DNA recognition by the Cas9 endonuclease. Nature 513, 569-573 (2014).
17. Weinberg Z, Perreault J, Meyer M M, Breaker R R, Exceptional structured noncoding RNAs revealed by bacterial metagenome analysis. Nature 462, 656-659 (2009).
18. Jumper J et al., Highly accurate protein structure prediction with AlphaFold. Nature 596, 583-589 (2021).
19. Sternberg S H, LaFrance B, Kaplan M, Doudna J A, Conformational control of DNA target cleavage by CRISPR-Cas9. Nature 527, 110-113 (2015).
20. Gong S, Yu H H, Johnson K A, Taylor D W, DNA Unwinding Is the Primary Determinant of CRISPR-Cas9 Activity. Cell reports 22, 359-371 (2018).
21. Workman R E et al., A natural single-guide RNA repurposes Cas9 to autoregulate CRISPR-Cas expression. Cell 184, 675-688 e619 (2021).
22. Mir A et al., Heavily and fully modified RNAs guide efficient SpyCas9-mediated genome editing. Nat Commun 9, 2641 (2018).
23. Gaudelli N M et al., Programmable base editing of A*T to G*C in genomic DNA without DNA cleavage. Nature 551, 464-471 (2017).
24. Anzalone A V et al., Search-and-replace genome editing without double-strand breaks or donor DNA. Nature 576, 149-157 (2019).
25. Komor A C, Kim Y B, Packer M S, Zuris J A, Liu D R, Programmable editing of a target base in genomic DNA without double-stranded DNA cleavage. Nature 533, 420-424 (2016).
26. Anzalone A V et al., Programmable deletion, replacement, integration and inversion of large DNA sequences with twin prime editing. Nat Biotechnol, (2021).
27. Ioannidi E I et al., Drag-and-drop genome insertion without DNA cleavage with CRISPR-directed integrases. bioRxiv, 2021.2011.2001.466786 (2021).
28. Punjani A, Rubinstein J L, Fleet D J, Brubaker M A, cryoSPARC: algorithms for rapid unsupervised cryo-EM structure determination. Nat Methods 14, 290-296 (2017).

1. A protein that is functional in RNA-guided DNA cleavage, wherein the protein comprises:
i) a modified IscB protein comprising a modification of its N-terminus, wherein the modification comprises a truncation of amino acids from the N-terminus which optionally comprises a PLMP domain; or
ii) a modified IscB protein that comprises a PLMP domain that is positioned at a location that is not the N-terminus.

2. The protein of claim 1, comprising the truncation of amino acids.

3. The protein of claim 2, wherein the truncation of amino acids comprises the PLMP domain.

4. The protein of claim 1, comprising the PLMP domain that is positioned at a location that is not the N-terminus.

5. The protein of claim 4, wherein the PLMP domain is positioned at the C-terminus, and wherein the protein optionally comprises a linker amino acid sequence that is connected to the PLMP domain.

6. The protein of claim 5, further comprising additional amino acids at the N-terminus, wherein the additional amino acids optionally comprise an enzyme, or a nucleic acid interaction domain.

7. The protein of claim 6, wherein the additional amino acids at the N-terminus comprise the enzyme.

8. The protein of claim 7, wherein the enzyme comprises a reverse transcriptase.

9. The protein of claim 1, wherein the protein comprises a segment that is at least 90% identical to SEQ ID NO:2 or SEQ ID NO:6.

10. The protein of claim 9, wherein the protein comprises at least one gain of function mutation, wherein the gain of function mutation is optionally selected from the mutations of Table A.

11. The protein of claim 1, wherein the protein comprises a nuclear localization signal.

12. A method comprising introducing into cells a protein of claim 1, and a ωRNA comprising a sequence targeted to target sequence within a polynucleotide sequence within the cell, such that the protein and the ωRNA locate to the target sequence.

13. The method of claim 12, wherein the protein and the ωRNA are introduced into the cell as a ribonucleoprotein (RNP).

14. The method of claim 13, wherein the protein, the ωRNA, or both, are introduced into the cell by expression from an expression vector.

15. The method of claim 14, wherein the expression vector is a recombinant adeno-associated virus (rAAV),

16. The method of claim 12, wherein the target sequence is modified by the protein.

17. The method of claim 12, wherein the cells are eukaryotic cells, and wherein the protein comprises at least one nuclear localization signal.

18. A cDNA or expression vector encoding a protein of claim 1.

19. A viral expression vector encoding a protein of claim 1.

20. An isolated complex comprising a protein of claim 1, the complex further comprising an ωRNA.

21. A cell comprising a protein of claim 1.

22. A system comprising a protein of claim 1, and a ωRNA that is functional with the protein.
